(19) United States
(12) Patent Application Publication
(10) Pub. No.: US 2020/0118645 A1
(43) Pub. Date: Apr. 16, 2020

(54) APPARATUSES AND METHODS OF CANCER CELL PREDICATION

(71) Applicant: SPES GENOMICS LTD., TAIPEI CITY (TW)

(72) Inventor: CHI-CHUN YEH, TAIPEI CITY (TW)

(21) Appl. No.: 16/654,037

(22) Filed: Oct. 16, 2019

Related U.S. Application Data
(60) Provisional application No. 62/746,187, filed on Oct. 16, 2018.

(51) Int. Cl. G16B 20/20; G16B 20/10; G16H 50/20

(52) U.S. Cl. CPC G16B 20/20; G16H 50/20; G16B 20/10

(57) ABSTRACT

An apparatus for prescription processing configured to perform: receiving a first to sixth values; determining a seventh to ninth values from a first function, the first to sixth values; determining a tenth to twelfth values from a second function, the first to sixth values; determining a first to sixth velocities from the seventh to twelfth values; determining a first set of coefficients from the first to sixth velocities and the seventh to twelfth values; determining a second set of coefficients from the first, second, fourth, and fifth velocities and the seventh, eighth, tenth, and eleventh values; for each of the first set of coefficients, determining a variance with the corresponding coefficient in the second set of coefficients; determining a first variance sum by summing the first and second sets of coefficients' variances; and saving the first variance sum, the first set of coefficients, the first and second functions.

### CROSS-REFERENCES TO RELATED APPLICATIONS

The present application claims priority from U.S. provisional patent application Ser. No. 62/746,187 filed Oct. 16, 2018; the disclosure of which is incorporated herein by reference in its entirety.

### FIELD OF THE INVENTION

The present invention relates to apparatuses and methods of cancer cell prediction. Particularly, the present invention provides an apparatus and a method to quantify the driver score of the cancer cells harboring different somatic genomic alterations (SGAs), predict the dynamics of the number of cancer cells, and predict the dynamics of the number of cancer cells in response to a subsequent SGA-targeted treatment at the individual-patient level.

### BACKGROUND OF THE INVENTION

The initiation and progression of cancers are driven by somatic genomic alterations (SGAs) that occurred in cancer genes. Cancer genes had been discovered and characterized by genetic, biochemical, clinical and large-scale cancer genome sequencing studies.

US 20150159220 discloses methods for predicting and detecting cancer risk using genetic markers such as somatic genomic alterations (SGA) that are associated with cancer risk. Also disclosed herein are methods for predicting and detecting a risk of esophageal adenocarcinoma (EA) based on the use of SGA that are associated with a risk of EA. US 20160203195 relates to a computer-implemented method for displaying and analyzing sequenced genome data, including obtaining, at a computing system, genomic data for a plurality of organisms. A graphical representation of the genomic data can be generated by the computing system based at least on the genomic data for the plurality of organisms, and the graphical representation can include a plurality of tracks that are arranged to show one or more features of the genomic data for different ones of the plurality of organisms. The graphical representation can be output for display by the computing system.

Clinical cancer sequencing assays provide the information about the SGAs of a cancer to physicians and patients. However, the information from current clinical cancer sequencing assays is insufficient for the effective clinical decision-making for the individual patient.

### SUMMARY

To provide a customized cancer cells prediction for individual patient, the present disclosure provides a novel model, a novel method, and an apparatus for performing the model and method. To provide a more accurate cancer cells prediction, the present disclosure provides a new method and an apparatus for performing the method. To provide a more accurate cancer cells prediction within a reasonable complexity, the present disclosure provides a new method an apparatus for performing the method.

In one aspect according to some embodiments, an apparatus comprises at least one non-transitory computer-readable medium and at least one processor. The at least one non-transitory computer-readable medium has computer executable instructions stored therein. The at least one processor is coupled to the at least one non-transitory computer-readable medium. The at least one non-transitory computer-readable medium and the computer executable instructions are configured to, with the at least one processor, cause the apparatus to perform: receiving a first value, a second value, and a third value; receiving a fourth value, a fifth value, and a sixth value; determining a seventh value based on a first function, the first value, and the fourth value; determining an eighth value based on the first function, the second value, and the fifth value; determining a ninth value based on the first function, the third value, and the sixth value; determining a tenth value based on a second function, the first value, and the fourth value; determining an eleventh value based on the second function, the second value, and the fifth value; determining a twelfth value based on the second function, the third value, and the sixth value; determining a first velocity, a second velocity, and a third velocity based on the seventh to ninth values; determining a fourth velocity, a fifth velocity, and a sixth velocity based on the tenth to twelfth values; determining a first set of coefficients based on the first to sixth velocities and the seventh to twelfth values; determining a second set of coefficients based on the first, second, fourth and fifth velocities and the seventh, eighth, tenth, and eleventh values; for each coefficient of the first set of coefficients, determining a variance with the corresponding coefficient in the second set of coefficients; determining a first variance sum by summing the variances for the coefficients in the first and second sets of coefficients; and saving the first variance sum, the first set of coefficients, the first function, and the second function. The first value indicates a detected count of cells having a first feature at a first time, the second value indicates a detected count of cells having a first feature at a second time, and the third value indicates a detected count of cells having a first feature at a third time. The fourth value indicates a detected count of cells having a second feature at the first time, the fifth value indicates a detected count of cells having the second feature at the second time, and the sixth value indicates a detected count of cells having the second feature at the third time

In one aspect according to some embodiments, an apparatus comprises at least one non-transitory computer-readable medium and at least one processor. The at least one non-transitory computer-readable medium has computer executable instructions stored therein. The at least one processor is coupled to the at least one non-transitory computer-readable medium. The at least one non-transitory computer-readable medium and the computer executable instructions are configured to, with the at least one processor, cause the apparatus to further perform: determining a thirteenth value based on a third function, the first value, and the fourth value; determining a fourteenth value based on the third function, the second value, and the fifth value; determining a fifteenth value based on the third function, the third value, and the sixth value; determining a sixteenth value based on a fourth function, the first value, and the fourth value; determining a seventeenth value based on the fourth function, the second value, and the fifth value; determining an eighteenth value based on the fourth function, the third value, and the sixth value; determining an seventh velocity, an eighth velocity, and a ninth velocity based on the thirteenth to fifteenth values; determining a tenth velocity, a eleventh velocity, and a twelfth velocity based on the sixteenth to eighteenth values; determining a third set of coefficients based on the seventh to twelfth velocities and the thirteenth to eighteenth values; determining a fourth set of coefficients based on the seventh, eighth, tenth, and eleventh velocities and the thirteenth, fourteenth, sixteenth, and seventeenth values; for each coefficient of the third set of coefficients, determining a variance with the corresponding coefficient in the fourth set of coefficients; determining a second variance sum by summing the variances for the coefficients in the third and fourth sets of coefficients; comparing the saved variance sum and the second variance sum; and if the saved variance sum is greater than the second variance sum, replacing the saved variance sum with the second variance sum, replacing the saved set of coefficients with the third set of coefficients, and replacing the saved functions with the third function and the fourth function.

In one aspect according to some embodiments, an apparatus comprises at least one non-transitory computer-readable medium and at least one processor. The at least one non-transitory computer-readable medium has computer executable instructions stored therein. The at least one processor is coupled to the at least one non-transitory computer-readable medium. The at least one non-transitory computer-readable medium and the computer executable instructions are configured to, with the at least one processor, cause the apparatus to perform: receiving first to ninth values; performing the operations disclosed in FIGS. 2A and 2B to determine a first matched type of a confounding factor between the first and second features; performing the operations disclosed in FIGS. 2A and 2B to determine a second matched type of the confounding factor between the first and third features; performing the operations disclosed in FIGS. 2A and 2B to determine a third matched type of the confounding factor between the second and third features; determining a first function, a second function, and a third function based on the first to third matched type of the confounding factor; determining a first accurate value based on the first function, the first value, the fourth value, and the seventh value; determining a second accurate value based on the first function, the second value, the fifth value, and the eighth value; determining a third accurate value based on the first function, the third value, the sixth value, and the ninth value; determining a fourth accurate value based on the second function, the first value, the fourth value, and the seventh value; determining a fifth accurate value based on the second function, the second value, the fifth value, and the eighth value; determining a sixth accurate value based on the second function, the third value, the sixth value, and the ninth value; determining a seventh accurate value based on the third function, the first value, the fourth value, and the seventh value; determining an eighth accurate value based on the third function, the second value, the fifth value, and the eighth value; determining a ninth accurate value based on the third function, the third value, the sixth value, and the ninth value; determining a first velocity, a second velocity, and a third velocity based on the first to third accurate values; determining a fourth velocity, a fifth velocity, and a sixth velocity based on the fourth to sixth accurate values; determining a seventh velocity, an eighth velocity, and a ninth velocity based on the seventh to ninth accurate values; and determining a first set of coefficients based on the first to ninth velocities and the first to ninth accurate values. The first value indicates a detected count of cells having a first feature at a first time, the second value indicates a detected count of cells having a first feature at a second time, and the third value indicates a detected count of cells having a first feature at a third time. The fourth value indicates a detected count of cells having a second feature at the first time, the fifth value indicates a detected count of cells having the second feature at the second time, and the sixth value indicates a detected count of cells having the second feature at the third time. The seventh value indicates a detected count of cells having a third feature at the first time, the eighth value indicates a detected count of cells having the third feature at the second time, and the ninth value indicates a detected count of cells having the third feature at the third time.

### BRIEF DESCRIPTION OF THE DRAWINGS

For a better understanding of the nature and objects of some embodiments of the present disclosure, reference should be made to the following detailed description taken in conjunction with the accompanying drawings. In the drawings, identical or functionally identical elements are given the same reference numbers unless otherwise specified.

FIG. 1 illustrates a system according to some embodiments of the present disclosure.

FIGS. 2A and 2B are flow charts of operations according to some embodiments of the present disclosure.

FIG. 3 is a flow chart of operations according to some embodiments of the present disclosure.

### DETAILED DESCRIPTION OF THE INVENTION

As used herein, the term “cancer gene” refers to any gene that drives the initiation and progression of cancer upon alterations of its sequence.

As used herein, the term “somatic genomic alteration”, or SGA, refers to the DNA sequence changes or aberrations that have accumulated in the genome of a cell during the lifetime of a subject. SGAs include point mutations, deletions, gene fusions, gene amplifications, translocations, copy number gain, copy number loss, copy-neutral loss of heterozygosity, homozygous deletion, and chromosomal rearrangements.

As used herein, the term “driver SGA” refers to any SGA that promotes the increase in the cancer cell number in a patient.

As used herein, the term “driver score” refers to the quantitative index of an SGA that indicates the capability of an SGA to promote the increase in the cancer cell number in a patient.

As used herein, the term “intraspecific competition” refers to the competition for the growth and survival between cancer cells those harbor the same SGA.

As used herein, the term “interspecific competition” refers to the competition for the growth and survival between cancer cells those harbor different SGAs.

As used herein, the term “dynamics of the abundance of SGA” refers to the temporal changes of the abundance of DNA molecules those harbor a given SGA in a cancer.

As used herein, the term “dynamics of the cancer cell number” refers to the temporal changes of the number of cancer cells in a patient.

As used herein, the term “somatic-genomic-alteration-targeted treatment (SGA-targeted treatment)” refers to a treatment that exerts its anti-cancer activity through inhibiting an SGA (or SGAs) specifically.

### The Quantification of the Driver Score.

Before the present disclosure, there is no effective cancer cell growth prediction method routinely used in clinical practice. The indices for describing treatment effects or prognosis information for patients includes, among others, tumor response rate, disease-free survival, overall survival, and five-year survival. These indices are population-level statistical information. For individual patients, these indices can only provide generally rough information. The present disclosure provides predictions information for individual patients and is helpful for clinical decisions.

The driver score of an SGA is quantified by the following method. A driver SGA, by definition, promotes the increase in the number of cancer cells those harbor it, so the abundance of the corresponding SGA increases over time. While applying the logistic growth function with the intraspecific and interspecific competition to the current method, the driver score of an SGA is broken down into three components: (1) the per capita growth rate of cancer cells harboring the SGA; (2) the parameter for sensitivity to the intraspecific competition of cancer cells those harbor the same SGA; and (3) the parameter for sensitivity to the interspecific competition of cancer cells those harbor different SGAs. The per capita growth rate of cancer cells may be represented as a summation of the cancer-cell-intrinsic effects of SGA on the cellular growth and survival, effects of anti-cancer treatment and effects of anti-cancer immunity on the cellular growth and survival. The intraspecific competition refers to the competition for the growth and survival between cancer cells those harbor the same SGA. The interspecific competition refers to the competition for the growth and survival between cancer cells those harbor different SGAs. An SGA that endows cancer cells with the higher per capita growth rate and the lower sensitivity to the intraspecific and interspecific competition has the higher driver score.

In one aspect, the present disclosure provides a process for monitoring the velocity of growth of cancer cells those harbor the SGAs under the intraspecific and interspecific competition and quantifying the driver score of the SGAs, comprising (i) determining the intrinsic velocity of growth of cancer cells harboring the SGAs; (ii) determining the effect of the intraspecific competition on the velocity of growth of cancer cells harboring the SGAs; and (iii) determining the effect of interspecific competition on the velocity of growth of cancer cells harboring the SGAs; whereby the velocity of growth of cancer cells those harbor the SGAs under the intraspecific and interspecific competition can be monitored and the driver score of the SGAs can be quantified.

In one embodiment, the velocity of growth of cancer cells those harbor the SGAs under the intraspecific and interspecific competition can be represented by the following differentiation equations:

$$\frac{{dN}_{1} ( t )}{dt} = {α}_{1} {N}_{1} ( t ) - {{β}_{1} [ {N}_{1} ( t ) ]}^{2} - {γ}_{12} {N}_{1} ( t ) {N}_{2} ( t )$$

$$\frac{{dN}_{2} ( t )}{dt} = {α}_{2} {N}_{2} ( t ) - {{β}_{2} [ {N}_{2} ( t ) ]}^{2} - {γ}_{21} {N}_{2} ( t ) {N}_{1} ( t )$$

Assuming there are cancer cells those harbor SGA₁ and cancer cell those harbor SGA₂ in a cancer, the differentiation equations that describe the velocity of growth of cancer cell harboring SGA₁ and SGA₂ are:

$$\frac{{dN}_{1} ( t )}{dt} = {α}_{1} {N}_{1} ( t ) - {{β}_{1} [ {N}_{1} ( t ) ]}^{2} - {γ}_{12} {N}_{1} ( t ) {N}_{2} ( t ) \\ \frac{{dN}_{2} ( t )}{dt} = {α}_{2} {N}_{2} ( t ) - {{β}_{2} [ {N}_{2} ( t ) ]}^{2} - {γ}_{21} {N}_{2} ( t ) {N}_{1} ( t ) \qquad Equation ( a )$$

whereas N₁(t) is the number of cancer cells those harbor SGA₁ at time-point t, N₂(t) is the number of cancer cells those harbor SGA₂ at time-point t; α₁ is the per capita growth rate of cancer cells those harbor SGA₁, α₂ is the per capita growth rate of cancer cells those harbor SGA₂; β₁ is the parameter for sensitivity to the intraspecific competition of cancer cells those harbor SGA₁, β₂ is the parameter for sensitivity to the intraspecific competition of cancer cells those harbor SGA₂; γ₁₂ is the parameter for sensitivity to the interspecific competition of cancer cells those harbor SGA₁ when competing with cancer cells those harbor SGA₂, γ₂₁ is the parameter for sensitivity to the interspecific competition of cancer cells those harbor SGA₂ when competing with cancer cells those harbor SGA₁; the time derivative of N₁(t) means the velocity of growth of cancer cells those harbor SGA₁, the time derivative of N₂(t) means the velocity of growth of cancer cells those harbor SGA₂.

In another aspect, the present disclosure provides a process for monitoring the velocity of growth of cancer cells when cancer cells those harbor different SGAs are competing with each other and quantifying the driver scores of those different SGAs, comprising (iv) determining the intrinsic velocity of growth of cancer cells harboring the SGAs; (v) determining the effect of the intraspecific competition on the velocity of growth of cancer cells harboring the SGAs; and (vi) determining the effect of the interspecific competition on the velocity of growth of cancer cells harboring the SGAs; whereby the velocity of growth of cancer cells when cancer cells those harbor different SGAs are competing with each other can be monitored and the driver scores of the SGAs can be quantified.

In one embodiment, the velocity of growth of cancer cells when cancer cells those harbor different SGAs are competing with each other can be represented by the following differentiation equation:

$$\frac{{dN}_{i} ( t )}{dt} = {α}_{i} {N}_{i} ( t ) - {{β}_{i} [ {N}_{i} ( t ) ]}^{2} - {\sum }_{j = 1}^{K} {γ}_{ij} {N}_{j} ( t ) {N}_{i} ( t ) \qquad equation ( b )$$

whereas Nᵢ(t) is the number of cancer cells those harbor SGAᵢ at timepoint t; αᵢ is the per capita growth rate of cancer cells those harbor SGAᵢ; βᵢ is the parameter for sensitivity to the intraspecific competition of cancer cells those harbor SGAᵢ; γᵢⱼ is the parameter for sensitivity to the interspecific competition of cancer cells those harbor SGA when competing with cancer cells those harbor SGAⱼ; the time derivative of Nᵢ(t) means the velocity of growth of cancer cells harbor SGA.

In one aspect, the present disclosure provides an apparatus for monitoring the cancer cell growth velocity and quantifying the driver score, comprising:

a receiving module configured to receive multiple signals of cancer cells harboring the SGAs corresponding multiple time points, wherein each signal represents the cancer cell number harboring a specific SGA at a time point;

a communication module configured to transmit the multiple values to a database;

a processor coupled with the communication module and configured to obtain the value of the per capital growth rate, the value of the parameter for sensitivity to the intraspecific competition, and the value of the parameter for sensitivity to the interspecific competition of cancer cells harboring the SGAs by applying statistical parameter estimation methods to estimate the values of the parameters of the following differentiation equation.

$$\frac{{dN}_{i} ( t )}{dt} = {α}_{i} {N}_{i} ( t ) - {{β}_{i} [ {N}_{i} ( t ) ]}^{2} - {\sum }_{j = 1}^{K} {γ}_{ij} {N}_{j} ( t ) {N}_{i} ( t ) ,$$

whereas Nᵢ(t) is the number of cancer cells those harbor SGAᵢ at timepoint t; αᵢ is the per capita growth rate of cancer cells those harbor SGAᵢ; βᵢ is the parameter for sensitivity to the intraspecific competition of cancer cells those harbor SGAᵢ; γᵢⱼ is the parameter for sensitivity to the interspecific competition of cancer cells those harbor SGA when competing with cancer cells those harbor SGAⱼ; the time derivative of Nᵢ(t) means the velocity of growth of cancer cells harbor SGAᵢ; and

a display module coupled with the processor and configured to display the value of per capita growth rate, the value of parameter for sensitivity to the intraspecific competition and the value of parameter for sensitivity to the interspecific competition of cancer cell harboring the SGAs.

In another aspect, the present disclosure provides a method for monitoring the velocity of growth of cancer cell and quantifying the driver score, comprising receive multiple signals of cancer cells harboring the SGAs corresponding multiple time points, wherein each signal represents the cancer cell number harboring a specific SGA at a time point; transmitting the multiple values to a database; obtaining the value of per capital growth rate, the value of parameter for sensitivity to the intraspecific competition and the value of parameter for sensitivity to the interspecific competition of cancer cells harboring the SGAs by applying statistical parameter estimation methods to estimate the values of the parameters of the following differentiation equation.

$$\frac{{dN}_{i} ( t )}{dt} = {α}_{i} {N}_{i} ( t ) - {{β}_{i} [ {N}_{i} ( t ) ]}^{2} - {\sum }_{j = 1}^{K} {γ}_{ij} {N}_{j} ( t ) {N}_{i} ( t ) ,$$

whereas Nᵢ(t) is the number of cancer cells those harbor SGAᵢ at timepoint t; αᵢ is the per capita growth rate of cancer cells those harbor SGAᵢ; βᵢ is the parameter for sensitivity to the intraspecific competition of cancer cells those harbor SGAᵢ; γᵢⱼ is the parameter for sensitivity to the interspecific competition of cancer cells those harbor SGAᵢ when competing with cancer cells those harbor SGAⱼ; the time derivative of Nᵢ(t) means the velocity of growth of cancer cells those harbor SGAᵢ; and displaying the value of the per capita growth rate, the value of the parameter for sensitivity to the intraspecific competition and the value of the parameter for sensitivity to the interspecific competition of cancer cells harboring the SGAs.

In one embodiment, the database is included in a server external to the apparatus and is used to store the first data and the second data.

In one embodiment, the database is built in the apparatus and is used to store the first data and the second data.

Given that αᵢ is associated with (1) the cancer-cell-intrinsic effect of a SGAᵢ on the cellular growth and survival, (2) effects of anti-cancer treatment and (3) effects of anti-cancer immunity, for a particular patient and a particular treatment. The per capita growth rate αᵢ for cancer cells those harbor SGAᵢ is assumed to be a constant. The parameter, βᵢ, for sensitivity to the intraspecific competition of cancer cells those harbor SGAᵢ is assumed to be a constant. The parameter, γᵢⱼ, for sensitivity to the interspecific competition is assumed to be a constant when cancer cells those harbor SGAᵢ are competing with cancer cells those harbor SGAⱼ. The values of parameters αᵢ, βᵢ, γᵢⱼ can be estimated by finding the values those best fit the observed data of the dynamics of the cancer cell number and the dynamics of the abundance of SGAᵢ by estimation methods with statistical criteria, such as the minimization of sum of squared errors or minimization of absolute percentage errors. Also, the values of parameter α₁, βᵢ, γᵢⱼ can be estimated by the Bayesian methods. Thus, three components of the driver score, α₁, βᵢ, γᵢⱼ can be quantified at the individual-patient level.

For example, for an individual patient, three to six examinations for SGA abundance and tumor image are taken within one to three months. Thus, an apparatus having at least one non-transitory computer-readable medium and at least one processor obtains numbers of cancer cells harboring different SGA at different times. For the example of examinations for two SGAs three times, an apparatus having at least one non-transitory computer-readable medium and at least one processor obtains N₁(t₁), N₁(t₂), N₁(t₃), N₂(t₁), N₂(t₂), and N₂(t₃). The time derivatives of the number of cancer cells harboring SGA₁ and SGA₂ at times t₁, t₂, and t₃

$$( i . e . , \frac{{N}_{1} ( {t}_{1} )}{dt} , \frac{{N}_{1} ( {t}_{2} )}{dt} , \frac{{N}_{1} ( {t}_{3} )}{dt} , \frac{{N}_{2} ( {t}_{1} )}{dt} , \frac{{N}_{2} ( {t}_{2} )}{dt} , \frac{{N}_{2} ( {t}_{3} )}{dt} ) ,$$

can be determined by the apparatus having at least one non-transitory computer-readable medium and at least one processor.

$$\frac{{N}_{1} ( {t}_{1} )}{dt}$$

can be regarded as the velocity of growth of cancer cells those harbor SGA₁ at time t₁ or the instantaneous velocity of growth of cancer cells those harbor SGA₁ at time t₁. According to Equation (a), the velocity of growth of cancer cell harboring SGA₁ and SGA₂ can be described below:

$$\frac{{dN}_{1} ( t )}{dt} = {α}_{1} {N}_{1} ( t ) - {{β}_{1} [ {N}_{1} ( t ) ]}^{2} - {γ}_{12} {N}_{2} ( t ) {N}_{1} ( t ) and$$

$$\frac{{dN}_{2} ( t )}{dt} = {α}_{2} {N}_{2} ( t ) - {{β}_{2} [ {N}_{2} ( t ) ]}^{2} - {γ}_{21} {N}_{1} ( t ) {N}_{2} ( t ) .$$

The apparatus having at least one non-transitory computer-readable medium and at least one processor determines coefficients α₁, β₁, γ₁₂, α₂, β₂, and γ₂₁ by finding the values (e.g., using the minimization of sum of squared errors or minimization of absolute percentage errors) those best fit the equations below:

$$\frac{{dN}_{1} ( {t}_{1} )}{dt} = {α}_{1} {N}_{1} ( {t}_{1} ) - {{β}_{1} [ {N}_{1} ( {t}_{1} ) ]}^{2} - {γ}_{12} {N}_{2} ( {t}_{1} ) {N}_{1} ( {t}_{1} ) ;$$

$$\frac{{dN}_{1} ( {t}_{2} )}{dt} = {α}_{1} {N}_{1} ( {t}_{2} ) - {{β}_{1} [ {N}_{1} ( {t}_{2} ) ]}^{2} - {γ}_{12} {N}_{2} ( {t}_{2} ) {N}_{1} ( {t}_{2} ) ;$$

$$\frac{{dN}_{1} ( {t}_{3} )}{dt} = {α}_{1} {N}_{1} ( {t}_{3} ) - {{β}_{1} [ {N}_{1} ( {t}_{3} ) ]}^{2} - {γ}_{12} {N}_{2} ( {t}_{3} ) {N}_{1} ( {t}_{3} ) ;$$

$$\frac{{dN}_{2} ( {t}_{1} )}{dt} = {α}_{2} {N}_{2} ( {t}_{1} ) - {{β}_{2} [ {N}_{2} ( {t}_{1} ) ]}^{2} - {γ}_{21} {N}_{1} ( {t}_{1} ) {N}_{2} ( {t}_{1} ) ;$$

$$\frac{{dN}_{2} ( {t}_{2} )}{dt} = {α}_{2} {N}_{2} ( {t}_{2} ) - {{β}_{2} [ {N}_{2} ( {t}_{2} ) ]}^{2} - {γ}_{21} {N}_{1} ( {t}_{2} ) {N}_{2} ( {t}_{2} ) ; and$$

$$\frac{{dN}_{2} ( {t}_{3} )}{dt} = {α}_{2} {N}_{2} ( {t}_{3} ) - {{β}_{2} [ {N}_{2} ( {t}_{3} ) ]}^{2} - {γ}_{21} {N}_{1} ( {t}_{3} ) {N}_{2} ( {t}_{3} ) .$$

Once the coefficients α₁, β₁, γ₁₂, α₂, β₂, and γ₂₁ are determined, the apparatus having at least one non-transitory computer-readable medium and at least one processor generates the differentiation equations that describe the velocity of growth of cancer cell harboring SGA₁ and SGA₂.

For a further example of examinations for k SGAs six times, an apparatus having at least one non-transitory computer-readable medium and at least one processor obtains N₁(t₁) to N₁(t₆), N₂(t₁) to N₂(t₆), . . . , and Nₖ(t₁) to Nₖ(t₆). The apparatus determines the time derivatives of the number of cancer cells harboring SGA₁ to SGAₖ at t₁ to t₆. For example,

$$\frac{{N}_{1} ( {t}_{1} )}{dt} = {α}_{1} {N}_{1} ( {t}_{1} ) - {{β}_{1} [ {N}_{1} ( {t}_{1} ) ]}^{2} - {\sum }_{j = 1 , j \neq 1}^{k} {γ}_{1 j} {N}_{j} ( {t}_{1} ) {N}_{1} ( {t}_{1} )$$

$$⋮$$

$$\frac{{N}_{1} ( {t}_{6} )}{dt} = {α}_{1} {N}_{1} ( {t}_{6} ) - {{β}_{1} [ {N}_{1} ( {t}_{6} ) ]}^{2} - {\sum }_{j = 1 , j \neq 1}^{k} {γ}_{1 j} {N}_{j} ( {t}_{6} ) {N}_{1} ( {t}_{6} )$$

$$\frac{{N}_{2} ( {t}_{1} )}{dt} = {α}_{2} {N}_{2} ( {t}_{1} ) - {{β}_{2} [ {N}_{2} ( {t}_{1} ) ]}^{2} - {\sum }_{j = 1 , j \neq 2}^{k} {γ}_{2 j} {N}_{j} ( {t}_{1} ) {N}_{2} ( {t}_{1} )$$

$$⋮$$

$$\frac{{N}_{2} ( {t}_{6} )}{dt} = {α}_{2} {N}_{1} ( {t}_{6} ) - {{β}_{1} [ {N}_{2} ( {t}_{6} ) ]}^{2} - {\sum }_{j = 1 , j \neq 2}^{k} {γ}_{2 j} {N}_{j} ( {t}_{6} ) {N}_{2} ( {t}_{6} )$$

$$⋮$$

$$⋮$$

$$\frac{{N}_{k} ( {t}_{1} )}{dt} = {α}_{k} {N}_{k} ( {t}_{1} ) - {{β}_{k} [ {N}_{k} ( {t}_{1} ) ]}^{2} - {\sum }_{j = 1 , j \neq k}^{k} {γ}_{kj} {N}_{j} ( {t}_{1} ) {N}_{k} ( {t}_{1} )$$

$$⋮$$

$$\frac{{N}_{k} ( {t}_{6} )}{dt} = {α}_{k} {N}_{k} ( {t}_{6} ) - {{β}_{k} [ {N}_{k} ( {t}_{6} ) ]}^{2} - {\sum }_{j = 1 , j \neq k}^{k} {γ}_{kj} {N}_{j} ( {t}_{6} ) {N}_{k} ( {t}_{6} )$$

can be regarded as the velocity of growth of cancer cells those harbor SGA₁ at time t₁ or the instantaneous velocity of growth of cancer cells those harbor SGA₁ at time t₁. According to Equation (b), the apparatus determines the coefficients α₁ to αₖ, β₁ to βₖ, and γᵢⱼ (where i=1 to k, j=1 to k, and i≠j) by finding the values (e.g., using the minimization of sum of squared errors or minimization of absolute percentage errors) those best fit the equations below:

$$\frac{{dN}_{i} ( t )}{dt} = {α}_{i} {N}_{i} ( t ) - {{β}_{i} [ {N}_{i} ( t ) ]}^{2} - {\sum }_{j = 1 , j \neq i}^{K} {γ}_{ij} {N}_{j} ( t ) {N}_{i} ( t ) .$$

Once the coefficients α₁ to αₖ, β₁ to βₖ, and γᵢⱼ (where i=1 to k, j=1 to k, and i≠j) are determined, the apparatus generates the differentiation equations that describe the velocity of growth of cancer cell harboring SGA₁ to SGAₖ.

The differentiation equations that describe the velocity of growth of cancer cell harboring SGAᵢ may be represented as:

$$\frac{{dN}_{i} ( t )}{dt} = {α}_{i} {N}_{i} ( t ) - {{β}_{i} [ {N}_{i} ( t ) ]}^{2} - {\sum }_{j = 1 , j \neq i}^{k} {γ}_{ij} {N}_{j} ( t ) {N}_{i} ( t ) .$$

The factors that influence the velocity of growth of the cancer cells in a human body are three: (1) the mechanism that controls growth and death of cancer cells; (2) the competition between the same kind of cancer cells in a microenvironment; and (3) the competition between different kinds of cancer cells in a microenvironment.

Regarding the first factor, normal cells have a mechanism that controls the growth and death thereof. Cancer cells change such mechanism, and the velocities of growth and death of cancer cells are different normal cells. The mechanism that controls the growth and death of cancer cells determines the intrinsic velocity of growth of the cancer cells.

In one embodiment, the intrinsic velocity of growth is derived by multiplying the per capita growth rate of cancer cells those harbor the SGA by the number of cancer cells those harbor the SGA (e.g., αᵢNᵢ(t)), wherein the per capita growth rate (e.g., αᵢ) is defined as the increase in the number of cells per unit of time per unit of cell number. The per capita growth rate is determined by the biological effects of an SGA (or SGAs) on the growth and survival of cancer cells those harbor it (or them).

The second factor is described here. In human body, because of the limits of oxygen supply, nutrient supply, survival space, and the velocity of waste removal, the microenvironment has a load limit. The number of normal cells would not approach the load limit of a microenvironment because of the mechanism that controls the growth and death thereof. However for cancer cells, because the mechanism that controls the growth and death of cancer cells is changed, the velocity of growth of the cancer cells increases much, and the number of cancer cells approaches the load limit of an microenvironment. Even though the velocity of growth of the cancer cells increases because of the changes of mechanism, the velocity of growth of the cancer cells will decrease when the number of cancer cells approaches the load limit of the microenvironment. The decrease of the velocity of growth of the cancer cells due to the load limit of the microenvironment determines the effect of the intraspecific competition on the velocity of growth.

In one embodiment, the effect of the intraspecific competition on the velocity of growth is derived by multiplying the magnitude of the intraspecific competition by the parameter for sensitivity to the intraspecific competition (e.g., βᵢ[Nᵢ(t)]²). The magnitude of the intraspecific competition is represented by the number of cancer cells those harbor the SGA squared (e.g., [Nᵢ(t)]²). The parameter for sensitivity to the intraspecific competition is determined by the interactions between cancer cells those harbors the same SGA and the interactions between cancer cells and the load limit of the microenvironment in which they reside.

Regarding the third factor, the cancer cells harboring different SGAs may have different sensitivities to the load limit of the microenvironment in which they reside. Thus, the cancer cells harboring different SGAs compete with each other. For example, if a SGA gives cancer cells the ability of surviving in a low-oxygen microenvironment; such cancer cells have survival advantage in the microenvironment with limited oxygen. The different sensitivities to the load limit of the cancer cells harboring different SGAs determine the effect of the interspecific competition on the velocity of growth.

In one embodiment, the effect of the interspecific competition on the velocity of growth is derived by multiplying the magnitude of interspecific competition by the parameter for sensitivity to the interspecific competition (e.g., Σ_{j=1,j≠i}^{k}γᵢⱼNⱼ(t)Nᵢ(t)). The magnitude of the interspecific competition between cancer cells those harbor two different SGAs is represented by multiplying the number of cancer cells those harbor the first SGA by the number of cancer cells those harbor the second SGA (e.g., Nⱼ(t)Nᵢ(t)).

In physiology, biochemistry, and molecular biology, relative researches of the three factors that influence the velocity of growth of the cancer cells in a human body can be found. However, such researches in physiology, biochemistry, and molecular biology are descriptions in properties or in vitro experiments. A method or apparatus of describing or predicting the number of cancer cells of an individual cancer patient through quantifying techniques is not provided before the present disclosure.

According to Equations (b),

$$\frac{{dN}_{i} ( t )}{dt} = {α}_{i} {N}_{i} ( t ) - {{β}_{i} [ {N}_{i} ( t ) ]}^{2} - {\sum }_{j = 1 , j \neq i}^{k} {γ}_{ij} {N}_{j} ( t ) {N}_{i} ( t ) ,$$

the first to third terms describe the first to third factors, respectively. The coefficient αᵢ of the first term indicates the changes of the mechanism that controls the growth and death of the cancer cells harboring SGAᵢ. For example, due to the changes of the mechanism that controls the growth and death of the cells, the growth of cancer cells does not need a growth factor (wherein the growth of normal cells needs a growth factor). The changes of the mechanism that controls the growth and death of the cells may cause changes of immune functions such that immune cells cannot attack cancer cells.

The coefficient βᵢ of the second term is determined by the space, oxygen, nutrient, and other factors of the microenvironment in which the cancer cells harboring SGAᵢ reside. If the cancer cell metastasis occurs, the space for the cancer cells increase, and the survival competition will be moderated so that the coefficient βᵢ decreases. If cancer cells cause angiogenesis, oxygen supply and nutrient supply increase, and the survival competition will be moderated so that the coefficient βᵢ decreases.

The coefficient γᵢⱼ of the third term indicates the survival competitions between the cancer cells harboring different SGAs under the load limit of a microenvironment. Since different SGAs cause different advantages for survival competitions, the competitions between the cancer cells harboring different SGA are independently described in the third term. The coefficient γᵢⱼ is determined by survival space, oxygen supply, nutrient supply, and “relative” growth advantages of the cancer cells harboring different SGAs associated with environmental limitations (e.g., limited survival space, limited oxygen supply, and limited nutrient supply). The factors influencing growth of cancer cells are complicated. There are competitions between cancer cells. The present disclosure takes different factors into consideration and quantifies these factors so as to obtain a more accurate prediction. Additionally, according to Equation (b) and the procedures of calculating the coefficients, a customized or personal cancer medical treatment is provided.

Further explanations of the intrinsic velocity of growth, the effect of the intraspecific competition on the velocity of growth, and the effect of the interspecific competition on the velocity of growth are provided herein. First, the intrinsic velocity of growth of cancer cells those harbors an SGA represents the velocity of growth in the absence of the intraspecific and interspecific competition. The per capita growth rate is determined by the biological effects of an SGA (or SGAs) on the growth and survival of cancer cells those harbor it (or them).

Second, the effect of the intraspecific competition means the effect of competition within cancer cells those harbor the same SGA on the velocity of growth. The effect of intraspecific competition on the velocity of growth is derived by multiplying the magnitude of the intraspecific competition by the parameter for sensitivity to the intraspecific competition. The magnitude of the intraspecific competition is defined as the number of cancer cells those harbor the SGA squared. The parameter for sensitivity to the intraspecific competition is determined by the interactions between cancer cells those harbors the same SGA and the interactions between cancer cells and the microenvironment in which they reside.

Third, the effect of the interspecific competition means the effect of the competition between cancer cells those harbor different SGAs on the velocity of growth. The effect of the interspecific competition on the velocity of growth is derived by multiplying the magnitude of the interspecific competition by the parameter for sensitivity to the interspecific competition. The magnitude of the interspecific competition between cancer cells those harbor two different SGAs is represented by multiplying the number of cancer cells those harbor the first SGA by the number of cancer cells those harbor the second SGA. The parameter for sensitivity to the intraspecific competition is determined by the interactions between cancer cells those harbors two different SGAs and the interactions between cancer cells and the microenvironment in which they reside. When cancer cells those harbor an SGA are competing with more than one cancer cell populations those harbor different SGAs, the effects of the interspecific competition between cancer cells those harbor different SGAs on the velocity of growth are summed to derive the total effect. In summary, the velocity of growth of cancer cells those harbor the SGAs is derived by subtracting intrinsic velocity of growth by the effect of the intraspecific competition and the effect of the interspecific competition on the velocity of growth.

In one embodiment, cancer cells those harbor SGAᵢ and cancer cells those harbor SGAⱼ are in a competition relationship if γᵢⱼ is a positive value and cancer cells those harbor SGAᵢ and cancer cells those harbor SGA_{J} are in a cooperative relationship if γᵢⱼ is a negative value.

In one embodiment, the per capita growth rate, the parameter for sensitivity to the intraspecific competition and the parameter for sensitivity to the interspecific competition of cancer cells harboring the SGAs relate to a forecast of the dynamics of the cancer cell and are used for indicating the subsequent treatment.

The relative abundance of an SGA is derived from examining the cancer-cell-derived genetic materials, such as DNA or RNA, obtained from the tumor tissue or bodily fluid. The examining methods may include PCR-based (Polymerase chain reaction-based) and/or sequencing-based approaches. The dynamics of the relative abundance of an SGA is obtained by serial examinations. The time-interval of examining tumor-derived genetic materials may be fixed or varied. The relative abundance of an SGA is represented as the fraction of DNA that carries the SGA in the total DNA that is examined. Assuming that the tumor volume is proportional to the cell number, the total cancer cell number of an individual patient is estimated by the tumor volume data obtained by image studies, such as the CT scan, PET scan, PET/CT scan, MRI scan, and sonographic scan. The dynamics of the total cancer cell number is obtained by serial image studies. The time-interval of serial image studies may be fixed or varied. The number of cancer cells that harbor an SGA is obtained by multiplying the total cancer cell number by the relative abundance of the corresponding SGA.

Correction of Confounding Factors in Deriving Accurate Number of Cancer Cells that Harbor a Specific SGA

The number of cancer cells that harbor a specific SGA (e.g., SGAᵢ) is obtained by multiplying the total cancer cell number (e.g., N_{T}(t)) by the relative abundance of the corresponding SGA (e.g., Aᵢ(t)). The confounding factors in deriving the accurate number of cancer cell that harbor a specific SGA should be addressed and corrected. The phylogenetic relationship (PR) of subclonal SGAs and the copy number (CN) of the gene that involved by the SGA are the two major confounding factors. An accurate PR of subclonal SGAs and an accurate CN of gene that involved by the SGA are essential in deriving the accurate number of cancer cells those harbor a specific SGA. The methods to determine the PR of subclonal SGAs and the CN of the gene that involved by the SGA are described in the following sections.

Description of the Phylogenetic Relationship of Subclonal SGAs and its Confounding Effect in Deriving the Accurate Number of Cancer Cell that Harbor a Specific SGA

Cancer is a clonal disease. A common ancestor cell gives rise to all cancer cells of a tumor. All cancer cells of the tumor will inherit the SGAs harbored by this common ancestor cell and these SGAs are called the “clonal SGAs”. On the other hand, any SGA occurred in cells other than the common ancestor cell will be inherited by the part, not all, of cancer cells of a tumor and these SGAs are called the “subclonal SGAs”. In order to describe the competition dynamics of subclonal SGAs correctly, it is essential to determine the PR of these subclonal SGAs. For any two subclonal SGAs, there are two possible types of the PR, either of the “parent-child” type relationship or of the “sibling” type relationship. A parent-child type of relationship between two SGAs refers to the relationship of two SGAs when one SGA (the child) occurred in a cell that already harbored the other SGA (the parent), which means that all cells those harbor the child SGA also harbor the parent SGA. A sibling type of relationship between two SGAs refers to the relationship of two SGAs when one SGA (the sibling 1) occurred in a cell that does not harbor the other SGA (the sibling 2), which means all cells those harbor sibling 1 SGA do not harbor sibling 2 SGA, and vice versa. The data of the abundance of an SGA represents a “sum-up” of an SGA from all cells those harbor the corresponding SGA. In the situation of the parent-child type of PR, the number of cells those harbor only the parent SGA is derived by subtracting the relative abundance of the parent SGA by the relative abundance of the child SGA (e.g., Aₚₐᵣₑₙₜ(t)−A_{child} (t), then multiplying by the total cancer cell number (e.g., N_{T}(t)×(Aₚₐᵣₑₙₜ(t)−A_{child}(t)) or Nₚₐᵣₑₙₜ(t)−N_{child}(t)). In the situation of the sibling type of PR, the number of cells those harbor either sibling SGA is derived from multiplying the relative abundance of either sibling SGA by the total cancer cell number (e.g., N_{T}(t)×A_{sibling 1}(t) and N_{T}(t)×A_{sibling 2}(t) or N_{sibling 1}(t) and N_{sibling 2}(t)). Thus, the determination of the accurate PR of subclonal SGAs is essential in deriving the accurate number of cancer cell that harbor a specific SGA.

### Description of the Copy Number of Genes Involved by an SGA and its Confounding Effect in Deriving the Accurate Number of Cancer Cells Those Harbor a Specific SGA

Humans are diploid organisms. There are two copies of each gene. An SGA may either involve only one or two copies of the gene. In the situation that an SGA involves two copies of the gene, and the number of cancer cells those harbor the corresponding SGA is corrected by dividing the original value by a factor of two

$$( e . g . , \frac{{N}_{T} ( t ) \times {A}_{i} ( t )}{2} or \frac{{N}_{i} ( t )}{2} ) .$$

In some situations, an SGA does not involves two copies of the gene, and the number of cancer cells those harbor the corresponding SGA is not corrected by dividing the original value by a factor of two (e.g., N_{T}(t)×Aᵢ(t) or Nᵢ(t)). Thus, the determination of the accurate CN of the gene involved by an SGA is essential in deriving the accurate number of cancer cell that harbor a specific SGA.

### Determination of the Phylogenetic Relationship of Subclonal SGAs and the Copy Number of Genes Involved by SGAs

In practices, it is hard to determine whether a SGA is clonal or subclonal through the current examining and experimental techniques. Furthermore, it is hard to determine whether an SGA involves two copies of the gene, in which a relative abundance of a SGA (e.g., Aᵢ(t)) has to be divided by two or not. It is also hard to determine whether a pair of SGAs belongs to the parent-child type of PR or the sibling type of PR.

A method or algorithm to generating the differentiation equations that describe the velocity of growth of cancer cell harboring different SGAs (e.g., Ń₁(t), Ń₂ (t), . . . , and Ńᵢ(t)), in which the confounding factors like PR and CN are considered, includes (1) determining, for all possible cases that PR and CN are considered, a first set of coefficients with all samples of the examination results (e.g., if the examinations for SGA abundance and tumor image were taken six times, the examination results for the six times are used) (2) determining, for all possible cases that PR and CN are considered, a second set of coefficients with a subset of the samples of the examination results (e.g., if the examinations for SGA abundance and tumor image were taken six times, the examination results for any three times are used), (3) for different possible cases, determining a variance sum of the coefficients in the first and second sets of coefficients, (4) determining which cases has the smallest variance sum, and (5) outputting the corresponding coefficients of the case having the smallest variance sum as having accurate numbers of cancer cells. Such method is called “sub-sample verification.” In some embodiments, the step (2) may be further performed one or more times with different subset of the samples to get more sets of coefficients, and the more sets of differentiation equations are used in step (3) to determine he variance sums of the coefficients in different cases. In some embodiments, an apparatus having at least one non-transitory computer-readable medium and at least one processor coupled to the at least one non-transitory computer-readable medium, wherein computer executable instructions are stored the at least one non-transitory computer-readable medium, and the computer executable instructions are configured to, with the at least one processor, cause the apparatus to perform the above method.

For an example of observing two SGAs by three examinations, the apparatus having at least one non-transitory computer-readable medium and at least one processor obtains N₁(t₁), N₁(t₂), N₁(t₃), N₂(t₁), N₂(t₂), and N₂(t₃). For N₁(t₁), N₁(t₂), N₁(t₃), N₂(t₁), N₂(t₂), and N₂(t₃), the confounding factors like PR and CN are not corrected yet. For every case that PR and CN are considered, the possible cases of accurate numbers of cancer cells harboring SGA₁ and SGA₂ in the three examinations, (i.e., Ń₁(t₁), Ń₁(t₂), Ń₁(t₃), Ń₂(t₁), Ń₂(t₂), Ń₂(t₃)), are listed below:

(i): N₁(t₁), N₁(t₂), N₁(t₃), N₂(t₁), N₂(t₂), N₂(t₃) (SGA₁ and SGA₂ belong to sibling type PR);
(ii):

$$\frac{{N}_{1} ( {t}_{1} )}{2} , \frac{{N}_{1} ( {t}_{2} )}{2} , \frac{{N}_{1} ( {t}_{3} )}{2} ,$$

N₂(t₁), N₂(t₂), N₂(t₃) (SGA₁ and SGA₂ belong to sibling type PR; SGA₁ involves CN);
(iii): N₁(t₁), N₁(t₂), N₁(t₃),

$$\frac{{N}_{2} ( {t}_{1} )}{2} , \frac{{N}_{2} ( {t}_{2} )}{2} , \frac{{N}_{2} ( {t}_{3} )}{2}$$

(SGA₁ and SGA₂ belong to sibling type PR; SGA₂ involves CN);
(iv):

$$\frac{{N}_{1} ( {t}_{1} )}{2} , \frac{{N}_{1} ( {t}_{2} )}{2} , \frac{{N}_{1} ( {t}_{3} )}{2} , \frac{{N}_{2} ( {t}_{1} )}{2} , \frac{{N}_{2} ( {t}_{2} )}{2} , \frac{{N}_{2} ( {t}_{3} )}{2}$$

(SGA₁ and SGA₂ belong to sibling type PR; SGA₁ and SGA₂ involve CN);
(v): N₁(t₁)−N₂(t₁), N₁(t₂)−N₂(t₂), N₁(t₃)−N₂(t₃), N₂(t₁), N₂(t₂), N₂(t₃) (SGA₁ to SGA₂ belong to parent-child type PR);
(vi):

$$\frac{{N}_{1} ( {t}_{1} )}{2} - {N}_{2} ( {t}_{1} ) , \frac{{N}_{1} ( {t}_{2} )}{2} - {N}_{2} ( {t}_{2} ) , \frac{{N}_{1} ( {t}_{3} )}{2} -- {N}_{2} ( {t}_{3} ) ,$$

N₂(t₁), N₂(t₂), N₂(t₃) (SGA₁ to SGA₂ belong to parent-child type PR; SGA₁ involves CN);
(vii):

$${N}_{1} ( {t}_{1} ) - \frac{{N}_{2} ( {t}_{1} )}{2} , {N}_{1} ( {t}_{2} ) - \frac{{N}_{2} ( {t}_{2} )}{2} , {N}_{1} ( {t}_{3} ) - \frac{{N}_{2} ( {t}_{3} )}{2} , \frac{{N}_{2} ( {t}_{1} )}{2} , \frac{{N}_{2} ( {t}_{2} )}{2} , \frac{{N}_{2} ( {t}_{3} )}{2}$$

(SGA₁ to SGA₂ belong to parent-child type PR; SGA₂ involves CN);
(viii):

$$\frac{{N}_{1} ( {t}_{1} )}{2} - \frac{{N}_{2} ( {t}_{1} )}{2} , \frac{{N}_{1} ( {t}_{2} )}{2} - \frac{{N}_{2} ( {t}_{2} )}{2} , \frac{{N}_{1} ( {t}_{3} )}{2} - \frac{{N}_{2} ( {t}_{3} )}{2} , \frac{{N}_{2} ( {t}_{1} )}{2} , \frac{{N}_{2} ( {t}_{2} )}{2} , \frac{{N}_{2} ( {t}_{3} )}{2}$$

(SGA₁ to SGA₂ belong to parent-child type PR; SGA₁ and SGA₂ involve CN);
(ix): N₁(t₁), N₁(t₂), N₁(t₃), N₂(t₁)−N₁(t₁), N₂(t₂)−N₁(t₂), N₂(t₃)−N₁(t₃) (SGA₂ to SGA₁ belong to parent-child type PR);
(x):

$$\frac{{N}_{1} ( {t}_{1} )}{2} , \frac{{N}_{1} ( {t}_{2} )}{2} , \frac{{N}_{1} ( {t}_{3} )}{2} , {N}_{2} ( {t}_{1} ) - \frac{{N}_{1} ( {t}_{1} )}{2} , {N}_{2} ( {t}_{2} ) - \frac{{N}_{1} ( {t}_{2} )}{2} , {N}_{2} ( {t}_{3} ) - \frac{{N}_{1} ( {t}_{3} )}{2}$$

(SGA₂ to SGA₁ belong to parent-child type PR; SGA₁ involves CN);
(xi): N₁(t₁), N₁(t₂), N₁(t₃),

$$\frac{{N}_{2} ( {t}_{1} )}{2} - {N}_{1} ( {t}_{1} ) , \frac{{N}_{2} ( {t}_{2} )}{2} - {N}_{1} ( {t}_{2} ) , \frac{{N}_{2} ( {t}_{3} )}{2} - {N}_{1} ( {t}_{3} )$$

(SGA₂ to SGA₁ belong to parent-child type PR; SGA₂ involves CN);
(xii):

$$\frac{{N}_{1} ( {t}_{1} )}{2} , \frac{{N}_{1} ( {t}_{2} )}{2} , \frac{{N}_{1} ( {t}_{3} )}{2} , \frac{{N}_{2} ( {t}_{1} )}{2} - \frac{{N}_{1} ( {t}_{1} )}{2} , \frac{{N}_{2} ( {t}_{2} )}{2} - \frac{{N}_{1} ( {t}_{2} )}{2} , \frac{{N}_{2} ( {t}_{3} )}{2} - \frac{{N}_{1} ( {t}_{3} )}{2}$$

(SGA₂ to SGA₁ belong to parent-child type PR; SGA₁ and SGA₂ involve CN).

Thus, for observing two SGAs, when PR and CN are considered, there are 12 possible cases of accurate numbers of cancer cells harboring SGA₁ and SGA₂. In one possible case, the accurate number of cancer cells harboring SGA₁ is determined by a function of the detected number of cancer cells harboring SGA₁ and the detected number of cancer cells harboring SGA₂, and the accurate number of cancer cells harboring SGA₂ is determined by another function of the detected number of cancer cells harboring SGA₁ and the detected number of cancer cells harboring SGA₂.

If any two SGAs are parent-child type PR, the number of cancer cells harboring the parent SGA is greater than the number cancer cells harboring the child SGA. In some embodiments, if the sequence of N₁(t₁), N₁(t₂), N₁(t₃) and the sequence of N₂(t₁), N₂(t₂), N₂(t₃) are crossed, the apparatus having at least one non-transitory computer-readable medium and at least one processor is caused to determine that SGA₁ and SGA₂ belong to sibling type PR rather than parent-child type PR or child-parent type PR. For example, if the relationships are N₁(t₁)>N₂(t₁), N₁(t₂)<N₂(t₂), and N₁(t₃)>N₂(t₃), the apparatus having at least one non-transitory computer-readable medium and at least one processor is caused to determine that SGA₁ and SGA₂ belong to sibling type PR. For another example, if the relationships are N₁(t₁)<N₂(t₁), N₁(t₂)>N₂(t₂), and N₁(t₃)<N₂(t₃), the apparatus having at least one non-transitory computer-readable medium and at least one processor is caused to determine that SGA₁ and SGA₂ belong to sibling type PR. Therefore, in some embodiments, when observing two SGAs, the possible cases considering PR and CN may less than 12. Furthermore, when considering other confounding factors, the possible to be analyzed between two SGAs may be less or more than 12.

For each of the 12 possible cases, the apparatus having at least one non-transitory computer-readable medium and at least one processor is caused to determine the time derivatives of the accurate number of cancer cells harboring SGA₁ and SGA₂ at times t₁ to t₃. That is, at different cases, the time derivative of the accurate number of cancer cells harboring SGA₁ and SGA₂ at t₁ to t₃ are determined from Ń₁(t₁), Ń₁(t₂), Ń₁(t₃), Ń₂(t₁), Ń₂(t₂), Ń₂(t₃). For example,

$$\frac{d {{N}^{′}}_{1} ( {t}_{1} )}{dt}$$

can be regarded as the velocity of growth of cancer cells those harbor SGA₁ at time t₁ or the instantaneous velocity of growth of cancer cells those harbor SGA₁ at time t₁. According to Equation (a), the apparatus is caused to determine the first set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₂₁¹ . . . γ₂₁¹² of the first set of coefficients (e.g., using the minimization of sum of squared errors or minimization of absolute percentage errors). For example, the apparatus is caused to determine the coefficients α₁¹, β₁¹, γ₁₂¹, α₂¹, β₂¹, and γ₂₁¹ by finding the values those best fit the equations below:

$$\frac{{dN}_{1} ( {t}_{1} )}{dt} = {α}_{1}^{1} {N}_{1} ( {t}_{1} ) - {{β}_{1}^{1} [ {N}_{1} ( {t}_{1} ) ]}^{2} - {γ}_{12}^{1} {N}_{2} ( {t}_{1} ) {N}_{1} ( {t}_{1} ) ;$$

$$\frac{{dN}_{1} ( {t}_{2} )}{dt} = {α}_{1}^{1} {N}_{1} ( {t}_{2} ) - {{β}_{1}^{1} [ {N}_{1} ( {t}_{2} ) ]}^{2} - {γ}_{12}^{1} {N}_{2} ( {t}_{2} ) {N}_{1} ( {t}_{2} ) ;$$

$$\frac{{dN}_{1} ( {t}_{3} )}{dt} = {α}_{1}^{1} {N}_{1} ( {t}_{3} ) - {{β}_{1}^{1} [ {N}_{1} ( {t}_{3} ) ]}^{2} - {γ}_{12}^{1} {N}_{2} ( {t}_{3} ) {N}_{1} ( {t}_{3} ) ;$$

$$\frac{{dN}_{2} ( {t}_{1} )}{dt} = {α}_{2}^{1} {N}_{2} ( {t}_{1} ) - {{β}_{2}^{1} [ {N}_{2} ( {t}_{1} ) ]}^{2} - {γ}_{21}^{1} {N}_{1} ( {t}_{1} ) {N}_{2} ( {t}_{1} ) ;$$

$$\frac{{dN}_{2} ( {t}_{2} )}{dt} = {α}_{2}^{1} {N}_{2} ( {t}_{2} ) - {{β}_{2}^{1} [ {N}_{2} ( {t}_{2} ) ]}^{2} - {γ}_{21}^{1} {N}_{1} ( {t}_{2} ) {N}_{2} ( {t}_{2} ) ;$$

$$\frac{{dN}_{2} ( {t}_{1} )}{dt} = {α}_{2}^{1} {N}_{2} ( {t}_{1} ) - {{β}_{2}^{1} [ {N}_{2} ( {t}_{3} ) ]}^{2} - {γ}_{21}^{1} {N}_{1} ( {t}_{3} ) {N}_{2} ( {t}_{3} ) .$$

The apparatus is caused to determine the coefficients α₁¹⁰, β₁¹⁰, γ₁₂¹⁰, α₂¹⁰, β₂¹⁰, and γ₂₁¹⁰ by finding the values those best fit the equations below:

$$\frac{d [ {N}_{1} ( {t}_{1} ) / 2 ]}{dt} = {α}_{1}^{1} \frac{{N}_{1} ( {t}_{1} )}{2} - {{β}_{1}^{1} [ \frac{{N}_{1} ( {t}_{1} )}{2} ]}^{2} - {γ}_{12}^{1} [ {N}_{2} ( {t}_{1} ) - \frac{{N}_{1} ( {t}_{1} )}{2} ] \frac{{N}_{1} ( {t}_{1} )}{2} ;$$

$$\frac{d [ {N}_{1} ( {t}_{2} ) / 2 ]}{dt} = {α}_{1}^{1} \frac{{N}_{1} ( {t}_{2} )}{2} - {{β}_{1}^{1} [ \frac{{N}_{1} ( {t}_{2} )}{2} ]}^{2} - {γ}_{12}^{1} [ {N}_{2} ( {t}_{2} ) - \frac{{N}_{1} ( {t}_{2} )}{2} ] \frac{{N}_{1} ( {t}_{2} )}{2} ;$$

$$\frac{d [ {N}_{1} ( {t}_{3} ) / 2 ]}{dt} = {α}_{1}^{1} \frac{{N}_{1} ( {t}_{3} )}{2} - {{β}_{1}^{1} [ \frac{{N}_{1} ( {t}_{3} )}{2} ]}^{2} - {γ}_{12}^{1} [ {N}_{2} ( {t}_{3} ) - \frac{{N}_{1} ( {t}_{3} )}{2} ] \frac{{N}_{1} ( {t}_{3} )}{2} ;$$

$$\frac{d [ {N}_{2} ( {t}_{1} ) - {N}_{1} ( {t}_{1} ) / 2 ]}{dt} = {α}_{2}^{1} [ {N}_{2} ( {t}_{1} ) - \frac{{N}_{1} ( {t}_{1} )}{2} ] - {{β}_{2}^{1} [ {N}_{2} ( {t}_{1} ) - \frac{{N}_{1} ( {t}_{1} )}{2} ]}^{2} - {γ}_{21}^{1} \frac{{N}_{1} ( {t}_{1} )}{2} [ {N}_{2} ( {t}_{1} ) - \frac{{N}_{1} ( {t}_{1} )}{2} ] ;$$

$$\frac{d [ {N}_{2} ( {t}_{2} ) - {N}_{1} ( {t}_{2} ) / 2 ]}{dt} = {α}_{2}^{1} [ {N}_{2} ( {t}_{2} ) - \frac{{N}_{1} ( {t}_{2} )}{2} ] - {{β}_{2}^{1} [ {N}_{2} ( {t}_{2} ) - \frac{{N}_{1} ( {t}_{2} )}{2} ]}^{2} - {γ}_{21}^{1} \frac{{N}_{1} ( {t}_{2} )}{2} [ {N}_{2} ( {t}_{2} ) - \frac{{N}_{1} ( {t}_{2} )}{2} ] ;$$

$$\frac{d [ {N}_{2} ( {t}_{3} ) - {N}_{1} ( {t}_{3} ) / 2 ]}{dt} = {α}_{2}^{1} [ {N}_{2} ( {t}_{3} ) - \frac{{N}_{1} ( {t}_{3} )}{2} ] - {{β}_{2}^{1} [ {N}_{2} ( {t}_{3} ) - \frac{{N}_{1} ( {t}_{3} )}{2} ]}^{2} - {γ}_{21}^{1} \frac{{N}_{1} ( {t}_{3} )}{2} [ {N}_{2} ( {t}_{3} ) - \frac{{N}_{1} ( {t}_{3} )}{2} ] .$$

For the second set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₂₁¹ . . . γ₂₁¹², they are determined or generated with a subset of the samples of the examination results by an apparatus having at least one non-transitory computer-readable medium and at least one processor. For example, the apparatus determines or generates the second set differentiation equations may be determined and generated with N₁(t₁), N₁(t₂), N₂(t₁), N₂(t₂), with N₁(t₁), N₁(t₃), N₂(t₁), N₂(t₃), or with N₁(t₂), N₁(t₃), N₂(t₂), N₂(t₃). Similar to the first set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₂₁¹ . . . γ₂₁¹², the apparatus estimates the second set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₁₂¹ . . . γ₂₁¹² according to Equation (a).

A set X has n values, x₁, x₂, . . . , xₙ. The variance of the set X can be written as

$$Var ( X ) = \frac{1}{n} {\sum }_{i = 1}^{n} {( {x}_{i} - μ )}^{2} ,$$

in which μ is the average value of the set X

$$( i . e . , μ = \frac{1}{n} {\sum }_{i = 1}^{n} {x}_{i}$$

One variance can be determined from α₁¹ in the first and second sets of coefficients. One variance can be determined from β₁¹ in the first and second sets of coefficients. One variance can be determined from γ₁₂¹ in the first and second sets of coefficients. One variance can be determined from al in the first and second sets of coefficients. One variance can be determined from β₂¹ in the first and second sets of coefficients. One variance can be determined from γ₂₁¹ in the first and second sets of coefficients. Thus, for the first possible case, there are six variances for the coefficients (i.e., Var(α₁¹), Var(β₁¹), Var(γ₁₂¹), Var(α₂¹), Var(β₂¹), and Var(γ₂₁¹)), and the variance sum for the first case is the summation of these six variances (i.e., Var(α₁¹)+Var(β₁¹)+Var(γ₁₂¹)+Var(α₂¹)+Var(β₂¹)+Var(γ₂₁¹)). Similarly, for each one of the other eleven possible cases, there are six variances for the coefficients, and the variance sum for each case is the summation of these six variances (i.e., Var(α₁²)+Var(β₁²)+Var(γ₁₂²)+Var(α₂²)+Var(β₂²)+Var(γ₂₁²), . . . . , Var(α₁¹²)+Var(β₁¹²)+Var(γ₁₂¹²)+Var(α₂¹²)+Var(β₂¹²)+Var(γ₂₁¹²)).

Through comparisons between the variance sums for the 12 cases by the apparatus having at least one non-transitory computer-readable medium and at least one processor, the case having the smallest variance sum can be determined by the apparatus. The apparatus determines the case with the smallest variance sum as the best guess. The apparatus outputs the corresponding coefficients of the case having the smallest variance sum (e.g., α₁¹⁰, β₁¹⁰, γ₁₂¹⁰, α₂¹⁰, β₂¹⁰, and γ₂₁¹⁰).

In some embodiments, an apparatus having at least one non-transitory computer-readable medium and at least one processor determines or generates the third set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₂₁¹ . . . γ₂₁¹² with another subset of the samples of the examination results. For example, if the apparatus determines or generates the second set of coefficients with N₁(t₁), N₁(t₂), N₂(t₁), N₂(t₂), the apparatus determines or generates the third set of coefficients with N₁(t₁), N₁(t₃), N₂(t₁), N₂(t₃), or with N₁(t₂), N₁(t₃), N₂(t₂), N₂(t₃).

In some embodiments, an apparatus having at least one non-transitory computer-readable medium and at least one processor determines or generates the fourth set of coefficients α₁¹ . . . α₁¹², β₁¹ . . . β₁¹², γ₁₂ . . . γ₁₂¹², α₂¹ . . . α₂¹², β₂¹ . . . β₂¹², and γ₂₁¹ . . . γ₂₁¹², with another subset of the samples of the examination results. For example, if the apparatus determines or generates the second set of coefficients with N₁(t₁), N₁(t₂), N₂(t₁), N₂(t₂), and if the apparatus determines or generates the third set of coefficients with N₁(t₁), N₁(t₃), N₂(t₁), N₂(t₃), the apparatus determines or generates the fourth set of coefficients with N₁(t₂), N₁(t₃), N₂(t₂), N₂(t₃).

In some embodiments that the third and fourth sets coefficients are determined or generated by an apparatus having at least one non-transitory computer-readable medium and at least one processor, one variance can be determined from α₁¹ in the first to fourth sets of coefficients; one variance can be determined from β₁¹ in the first to fourth sets of coefficients; one variance can be determined from γ₁₂¹ in the first to fourth sets of coefficients; one variance can be determined from α₂¹ in the first to fourth sets of coefficients; one variance can be determined from β₂¹ in the first to fourth sets of coefficients; and one variance can be determined from γ₂₁¹ in the first to fourth sets of coefficients. Thus, for the first possible case, there are six variances for the coefficients (i.e., Var(α₁¹), Var(β₁¹), Var(γ₁₂¹), Var(α₂¹), Var(β₂¹), and Var(γ₂₁¹), and the variance sum for the first case is the summation of these six variances (i.e., Var(α₁¹)+Var(β₁¹)+Var(γ₁₂¹)+Var(α₂¹)+Var(β₂¹)+Var(γ₂₁¹)). Similarly, for each one of the other eleven possible cases, there are six variances for the coefficients, and the variance sum for each case is the summation of these six variances (i.e., Var(α₁²)+Var(β₁²)+Var(γ₁₂²)+Var(α₂²)+Var(β₂²)+Var(γ₂₁²), . . . . , Var(α₁¹²)+Var(β₁¹²)+Var(γ₁₂¹²)+Var(α₂¹²)+Var(β₂¹²)+Var(γ₂₁¹²)).

Through comparisons the variance sums for the 12 cases by the apparatus having at least one non-transitory computer-readable medium and at least one processor, the case having the smallest variance sum can be determined by the apparatus. The apparatus determines the case with the smallest variance sum as the best guess. The apparatus then outputs the corresponding coefficients of the case with the smallest variance sum (e.g., α₁¹⁰, β₁¹⁰, γ₁₂¹⁰, α₂¹⁰, β₂¹⁰, and γ₂₁¹⁰). The best guess derived from the first to fourth sets of coefficients is more confidential than that derived from the first and second sets of coefficients.

If the best guess is the tenth case (when considering PR and CN), then the differentiation equations that describe the velocity of growth of cancer cell harboring SGA₁ and SGA₂ are given as

$${{N}^{'}}_{1} ( t ) = {α}_{1}^{10} {{N}^{'}}_{1} ( t ) - {{β}_{1}^{10} [ {{N}^{'}}_{1} ( t ) ]}^{2} - {γ}_{12}^{10} {{N}^{'}}_{2} ( t ) {{N}^{'}}_{1} ( t )$$

$$and \frac{{{N}^{'}}_{2} ( t )}{dt} = {α}_{2}^{10} {{N}^{'}}_{2} ( t ) - {{β}_{2}^{10} [ {{N}^{'}}_{2} ( t ) ]}^{2} - {γ}_{21}^{10} {{N}^{'}}_{1} ( t ) {{N}^{'}}_{2} ( t ) .$$

For an example of observing three SGAs by three examinations, the apparatus having at least one non-transitory computer-readable medium and at least one processor obtains N₁(t₁), N₁(t₂), N₁(t₃), N₂(t₁), N₂(t₂), N₂(t₃), N₃ (t₁), N₃ (t₂), and N₃ (t₃). For N₁(t₁), N₁(t₂), N₁(t₃), N₂(t₁), N₂(t₂), N₂(t₃), N₃ (t₁), N₃ (t₂), and N₃ (t₃), the confounding factors like PR and CN are not corrected yet. The number of possible cases considering PR and CN are derived below.

1. The possible cases based on PR are now considered. For three SGAs, there are C₂³

$$( i . e . , \frac{3 !}{2 ! ( 3 - 2 ) !} = 3 )$$

possible pairs of SGA. For any pair of SGAs, there are three possible types PR (i.e., parent-child type, child-parent type, and sibling type). If a pair of SGA belongs to parent-child type PR, the number of cancer cells harboring the first SGA is subtracted by the number of cancer cells harboring the second SGA (or the abundance of the first SGA is subtracted by the abundance of the second SGA). If a pair of SGA belongs to child-parent type PR, the number of cancer cells harboring the second SGA is subtracted by the number of cancer cells harboring the first SGA (or the abundance of the second SGA is subtracted by the abundance of the first SGA). If a pair of SGA belongs to sibling type PR, no subtraction is performed between the numbers of cancer cells harboring the first and second SGAs (or no subtraction is performed between the abundances of the first and second SGAs). Thus, based on PR, there are 3³ (i.e., 3^{c}^{₂}^{³}=27) possible cases.

2. The possible cases based on CN are now considered. For any SGA, there are two possible cases (i.e., involving CN or not involving CN). If one SGA involve CN, the number of cancer cells harboring the SGA (or the abundance of the SGA) is divided by two. If one SGA does not involve CN, the number of cancer cells harboring the SGA (or the abundance of the SGA) is not divided by two. Thus, based on CN, there are 2³ possible cases.

3. If PR and CN are considered, the number of possible cases for three SGAs is 3^{c}^{₂}^{³}×2³ (i.e., 216).

4. In general, when number of observed SGAs is n, the number of the possible cases can be derived as 3^{c}^{₂}^{ⁿ}×2ⁿ.

Thus, if 4 SGAs is observed, the number of possible cases is 3^{c}^{₂}^{⁴}×2⁴=729×16=11664. It can be understood that when the number of observed SGAs increases, the number of possible cases is exponentially increases, and the complexity of the above method or algorithm is too great. When observing n SGAs, he complexity is O(cⁿ), in which c is a constant. Furthermore, when 3 SGAs is observed, said 216 possible cases may not include that the first SGA is the parent of both the second and third SGAs. For an example that the numbers of the cancer cells harboring the first, second, and third SGAs are N₁, N₂, and N₃, if the first SGA the parent of both the second and third SGAs, the corrected number of cancer cells harboring the first SGA is N₁−N₂−N₃.

The present disclosure provide another method or algorithm to generating the differentiation equations that describe the velocity of growth of cancer cell harboring different SGAs (e.g., Ń₁(t), Ń₂(t), . . . , and Ńₗ(t)), in which the confounding factors like PR and CN are considered. The method and algorithm included (1) for the twelve cases of each pair of SGAs, determining a first set of coefficients with all samples of the examination results (e.g., if the examinations for SGA abundance and tumor image were taken six times, the examination results for the six times are used) (2) for the twelve cases of each pair of SGAs, determining a second set of coefficients with a subset of the samples of the examination results (e.g., if the examinations for SGA abundance and tumor image were taken six times, the examination results for any three times are used), (3) for different possible cases of each pair of SGAs, determining a variance sum of the coefficients in the first and second sets of coefficients, (4) for each pair of SGAs, determining which case has the smallest variance sum, (5) for each pair of SGAs, determining the type of PR according to the case having the smallest variance sum, (6) for each SGA, determining whether CN is involved according to the case having the smallest variance sum (7) for all of the observed SGAs, determining the accurate numbers of cancer cells harboring different SGA (e.g., Ń₁(t), Ń₂(t), . . . , and Ńₗ(t)) according to the type of PR and whether CN is involved, and (7) based on the accurate numbers of cancer cells harboring different SGAs, determining a set of prediction coefficients with all samples of the examination results. That is, for each pair of SGAs, the called “sub-sample verification” method is applied to determine the type of PR and whether CN is involved. In some embodiments, the step (2) may be further performed one or more times with different subset of the samples to get more sets of coefficients, and the more sets of differentiation equations are used in step (3) to determine the variance sums of the coefficients in different cases. In some embodiments, an apparatus having at least one non-transitory computer-readable medium and at least one processor coupled to the at least one non-transitory computer-readable medium, wherein computer executable instructions are stored the at least one non-transitory computer-readable medium, and the computer executable instructions are configured to, with the at least one processor, cause the apparatus to perform the above method.

For an example of observing three SGAs (i.e., SGA₁ to SGA₃) by four examinations, the apparatus having at least one non-transitory computer-readable medium and at least one processor obtains N₁(t₁) to N₁(t₄), N₂(t₁) to N₂(t₄), and N₃(t₁) to N₃ (t₄). For N₁(t₁) to N₁(t₄), N₂(t₁) to N₂(t₄), and N₃(t₁) to N₃(t₄), the confounding factors like PR and CN are not corrected yet. For each possible pair of SGA₁ to SGA₃, when PR and CN are considered, there are 12 possible cases. For example, when SGA₁ and SGA₂ are considered, the possible 12 cases of corrected numbers of cancer cells harboring SGA₁ and SGA₂ in the three examinations, (i.e., Ń₁(t₁), Ńᵢ(t₂), Ń₁(t₃), Ń₁(t₄), Ń₂(t₁), Ń₂(t₂), Ń₂(t₃), Ń₂(t₄)), are listed below:

(i): N₁(t₁), N₁(t₂), N₁(t₃), N₁(t₄), N₂(t₁), N₂(t₂), N₂(t₃), N₁(t₄) (SGA₁ and SGA₂ belong to sibling type PR);
(ii):

$$\frac{{N}_{1} ( {t}_{1} )}{2} , \frac{{N}_{1} ( {t}_{2} )}{2} , \frac{{N}_{1} ( {t}_{3} )}{2} , \frac{{N}_{1} ( {t}_{4} )}{2} ,$$

N₂(t₁), N₂(t₂), N₂(t₃), N₂(t₄) (SGA₁ and SGA₂ belong to sibling type PR; SGA₁ involves CN);
(iii): N₁(t₁), N₁(t₂), N₁(t₃), N₁(t₄),

$$\frac{{N}_{2} ( {t}_{1} )}{2} , \frac{{N}_{2} ( {t}_{2} )}{2} , \frac{{N}_{2} ( {t}_{3} )}{2} , \frac{{N}_{2} ( {t}_{4} )}{2} ,$$

(SGA₁ and SGA₂ belong to sibling type PR; SGA₂ involves CN);
(iv):

$$\frac{{N}_{1} ( {t}_{1} )}{2} , \frac{{N}_{1} ( {t}_{2} )}{2} , \frac{{N}_{1} ( {t}_{3} )}{2} , \frac{{N}_{1} ( {t}_{4} )}{2} , \frac{{N}_{2} ( {t}_{1} )}{2} , \frac{{N}_{2} ( {t}_{2} )}{2} , \frac{{N}_{2} ( {t}_{3} )}{2} , \frac{{N}_{2} ( {t}_{4} )}{2}$$

(SGA₁ and SGA₂ belong to sibling type PR; SGA₁ and SGA₂ involve CN);
(v): N₁(t₁)−N₂(t₁), N₁(t₂)−N₂(t₂), N₁(t₃)−N₂(t₃), N₁(t₄)−N₂(t₄), N₂(t₁), N₂(t₂), N₂(t₃), N₂(t₄) (SGA₁ to SGA₂ belong to parent-child type PR);
(vi):

$$\frac{{N}_{1} ( {t}_{1} )}{2} - {N}_{2} ( {t}_{1} ) , \frac{{N}_{1} ( {t}_{2} )}{2} - {N}_{2} ( {t}_{2} ) , \frac{{N}_{1} ( {t}_{3} )}{2} -- {N}_{2} ( {t}_{3} ) , \frac{{N}_{1 } ( {t}_{4} )}{2} -- {N}_{2} ( {t}_{4} ) ,$$

N₂(t₁), N₂(t₂), N₂(t₃), N₂(t₄) (SGA₁ to SGA₂ belong to parent-child type PR; SGA₁ involves CN);
(vii):

$${N}_{1} ( {t}_{1} ) - \frac{{N}_{2} ( {t}_{1} )}{2} , {N}_{1} ( {t}_{2} ) - \frac{{N}_{2} ( {t}_{2} )}{2} , {N}_{1} ( {t}_{3} ) - \frac{{N}_{2} ( {t}_{3} )}{2} , \\ {N}_{1} ( {t}_{4} ) - \frac{{N}_{2} ( {t}_{4} )}{2} , \frac{{N}_{2} ( {t}_{1} )}{2} , \frac{{N}_{2} ( {t}_{2} )}{2} , \frac{{N}_{2} ( {t}_{3} )}{2} , \frac{{N}_{2} ( {t}_{4} )}{2}$$

(SGA₁ to SGA₂ belong to parent-child type PR; SGA₂ involves CN);
(viii):

$$\frac{{N}_{1} ( {t}_{1} )}{2} - \frac{{N}_{2} ( {t}_{1} )}{2} , \frac{{N}_{1} ( {t}_{2} )}{2} - \frac{{N}_{2} ( {t}_{2} )}{2} , \frac{{N}_{1} ( {t}_{3} )}{2} - \frac{{N}_{2} ( {t}_{3} )}{2} , \frac{{N}_{1} ( {t}_{4} )}{2} - \frac{{N}_{2 } ( {t}_{4} )}{2} , \\ \frac{{N}_{2} ( {t}_{1} )}{2} , \frac{{N}_{2} ( {t}_{2} )}{2} , \frac{{N}_{2} ( {t}_{3} )}{2} , \frac{{N}_{2} ( {t}_{4} )}{2}$$

(SGA₁ to SGA₂ belong to parent-child type PR; SGA₁ and SGA₂ involve CN);
(ix): N₁(t₁), N₁(t₂), N₁(t₃), N₁(t₄), N₂(t₁)−N₁(t₁), N₂(t₂)−N₁(t₂), N₂(t₃)−N₁(t₃), N₂(t₄)−N₁(t₄) (SGA₂ to SGA₁ belong to parent-child type PR);
(x):

$$\frac{{N}_{1} ( {t}_{1} )}{2} , \frac{{N}_{1} ( {t}_{2} )}{2} , \frac{{N}_{1 } ( {t}_{3} )}{2} , \frac{{N}_{1} ( {t}_{4} )}{2} , {N}_{2} ( {t}_{1} ) - \frac{{N}_{1} ( {t}_{1} )}{2} , {N}_{2} ( {t}_{2} ) - \frac{{N}_{1} ( {t}_{2} )}{2} , \\ {N}_{2} ( {t}_{3} ) - \frac{{N}_{1} ( {t}_{3} )}{2} , {N}_{2} ( {t}_{4} ) - \frac{{N}_{1} ( {t}_{4} )}{2}$$

(SGA₂ to SGA₁ belong to parent-child type PR; SGA₁ involves CN);
(xi): N₁(t₁), N₁(t₂), N₁(t₃), N₁(t₄),

$$\frac{{N}_{2} ( {t}_{1} )}{2} - {N}_{1 } ( {t}_{1} ) , \frac{{N}_{2} ( {t}_{2} )}{2} - {N}_{1} ( {t}_{2} ) , \frac{{N}_{2} ( {t}_{3} )}{2} - {N}_{1} ( {t}_{3} ) , \frac{{N}_{2} ( {t}_{4} )}{2} - {N}_{1} ( {t}_{4} )$$

(SGA₂ to SGA₁ belong to parent-child type PR; SGA₂ involves CN);
(xii):

$$\frac{{N}_{1} ( {t}_{1} )}{2} , \frac{{N}_{1} ( {t}_{2} )}{2} , \frac{{N}_{1 } ( {t}_{3} )}{2} , \frac{{N}_{1} ( {t}_{4} )}{2} , \frac{{N}_{2} ( {t}_{1} )}{2} - \frac{{N}_{1} ( {t}_{1} )}{2} , \frac{{N}_{2} ( {t}_{2} )}{2} - \frac{{N}_{1 } ( {t}_{2} )}{2} , \\ \frac{{N}_{2} ( {t}_{3} )}{2} - \frac{{N}_{1} ( {t}_{3} )}{2} , \frac{{N}_{2} ( {t}_{4} )}{2} - \frac{{N}_{1} ( {t}_{4} )}{2}$$

(SGA₂ to SGA₁ belong to parent-child type PR; SGA₁ and SGA₂ involve CN).

Thus, observing SGA₁ and SGA₂, when PR and CN are considered, there are 12 possible cases of corrected numbers of cancer cells harboring SGA₁ and SGA₂. Observing SGA₁ and SGA₃, when PR and CN are considered, there are 12 possible cases of corrected numbers of cancer cells harboring SGA₁ and SGA₃. Observing SGA₂ and SGA₃, when PR and CN are considered, there are 12 possible cases of corrected numbers of cancer cells harboring SGA₂ and SGA₃.

When observing SGA₁ and SGA₂, in one possible case, the accurate number of cancer cells harboring SGA₁ is determined by a function of the detected number of cancer cells harboring SGA₁ and the detected number of cancer cells harboring SGA₂, and the accurate number of cancer cells harboring SGA₂ is determined by another function of the detected number of cancer cells harboring SGA₁ and the detected number of cancer cells harboring SGA₂. When observing SGA₁ and SGA₃, in one possible case, the accurate number of cancer cells harboring SGA₁ is determined by a function of the detected number of cancer cells harboring SGA₁ and the detected number of cancer cells harboring SGA₃, and the accurate number of cancer cells harboring SGA₃ is determined by another function of the detected number of cancer cells harboring SGA₁ and the detected number of cancer cells harboring SGA₃. When observing SGA₂ and SGA₃, in one possible case, the accurate number of cancer cells harboring SGA₂ is determined by a function of the detected number of cancer cells harboring SGA₂ and the detected number of cancer cells harboring SGA₃, and the accurate number of cancer cells harboring SGA₃ is determined by another function of the detected number of cancer cells harboring SGA₂ and the detected number of cancer cells harboring SGA₃.

If any two SGAs are parent-child type PR, the number of cancer cells harboring the parent SGA is greater than the number cancer cells harboring the child SGA. In some embodiments, if the sequence of N₁(t₁), N₁(t₂), N₁(t₃) and the sequence of N₂(t₁), N₂(t₂), N₂(t₃) are crossed, the apparatus having at least one non-transitory computer-readable medium and at least one processor is caused to determine that SGA₁ and SGA₂ belong to sibling type PR rather than parent-child type PR or child-parent type PR. For example, if the relationships are N₁(t₁)>N₂(t₁), N₁(t₂)<N₂(t₂), and N₁(t₃)>N₂(t₃), the apparatus having at least one non-transitory computer-readable medium and at least one processor is caused to determine that SGA₁ and SGA₂ belong to sibling type PR. For another example, if the relationships are N₁(t₁)<N₂(t₁), N₁(t₂)>N₂(t₂), and N₁(t₃)<N₂(t₃), the apparatus having at least one non-transitory computer-readable medium and at least one processor is caused to determine that SGA₁ and SGA₂ belong to sibling type PR. Therefore, in some embodiments, when observing a pair of SGAs, the possible cases considering PR and CN may less than 12. Furthermore, when considering other confounding factors, the possible to be analyzed between a pair of SGA may be less or more than 12.

For the 12 possible cases of SGA₁ and SGA₂, the apparatus having at least one non-transitory computer-readable medium and at least one processor is caused to determine time derivatives of the corrected number of cancer cells harboring SGA₁ to SGA₃ at times t₁ to t₄. For the example that the first case is considered,

$$\frac{{N}_{1} ( {t}_{1} )}{dt}$$

can be regarded as the velocity of growth of cancer cells those harbor SGA₁ at time t₁ or the instantaneous velocity of growth of cancer cells those harbor SGA₁ at time t₁. According to Equation (a), the apparatus is caused to determine the first set of coefficients α₁¹ . . . α₁¹², β₁¹ . . . β₁¹², γ₁₂¹ . . . γ₁₂¹², α₂¹ . . . β₂¹², β₂¹ . . . β₂¹², and γ₂₁¹ . . . γ₂₁¹² (e.g., using the minimization of sum of squared errors or minimization of absolute percentage errors). For example, the apparatus is caused to determine the coefficients α₁¹, β₁¹, γ₁₂¹, α₂¹, β₂¹, and γ₂₁¹ by finding the values those best fit the equations below:

$$\frac{{dN}_{1} ( {t}_{1} )}{dt} = {α}_{1}^{1} {N}_{1} ( {t}_{1} ) - {{β}_{1}^{1} [ {N}_{1} ( {t}_{1} ) ]}^{2} - {γ}_{12}^{1} {N}_{2} ( {t}_{1} ) {N}_{1} ( {t}_{1} ) ;$$

$$\frac{{dN}_{1} ( {t}_{2} )}{dt} = {α}_{1}^{1} {N}_{1} ( {t}_{2} ) - {{β}_{1}^{1} [ {N}_{1} ( {t}_{2} ) ]}^{2} - {γ}_{12}^{1} {N}_{2} ( {t}_{2} ) {N}_{1} ( {t}_{2} ) ;$$

$$\frac{{dN}_{1} ( {t}_{3} )}{dt} = {α}_{1}^{1} {N}_{1} ( {t}_{3} ) - {{β}_{1}^{1} [ {N}_{1} ( {t}_{3} ) ]}^{2} - {γ}_{12}^{1} {N}_{2} ( {t}_{3} ) {N}_{1} ( {t}_{3} ) ;$$

$$\frac{{dN}_{1} ( {t}_{4} )}{dt} = {α}_{1}^{1} {N}_{1} ( {t}_{4} ) - {{β}_{1}^{1} [ {N}_{1} ( {t}_{4} ) ]}^{2} - {γ}_{12}^{1} {N}_{2} ( {t}_{4} ) {N}_{1} ( {t}_{4} ) ;$$

$$\frac{{dN}_{2} ( {t}_{1} )}{dt} = {α}_{2}^{1} {N}_{2} ( {t}_{4} ) - {{β}_{2}^{1} [ {N}_{2} ( {t}_{1} ) ]}^{2} - {γ}_{21}^{1} {N}_{1} ( {t}_{1} ) {N}_{2} ( {t}_{1} ) ;$$

$$\frac{{dN}_{2} ( {t}_{2} )}{dt} = {α}_{2}^{1} {N}_{2} ( {t}_{2} ) - {{β}_{2}^{1} [ {N}_{2} ( {t}_{2} ) ]}^{2} - {γ}_{21}^{1} {N}_{1} ( {t}_{2} ) {N}_{2} ( {t}_{2} ) ;$$

$$\frac{{dN}_{2} ( {t}_{3} )}{dt} = {α}_{2}^{1} {N}_{2} ( {t}_{3} ) - {{β}_{2}^{1} [ {N}_{2} ( {t}_{3} ) ]}^{2} - {γ}_{21}^{1} {N}_{1} ( {t}_{3} ) {N}_{2} ( {t}_{3} ) ;$$

$$\frac{{dN}_{2} ( {t}_{4} )}{dt} = {α}_{2}^{1} {N}_{2} ( {t}_{4} ) - {{β}_{2}^{1} [ {N}_{2} ( {t}_{4} ) ]}^{2} - {γ}_{21}^{1} {N}_{1} ( {t}_{4} ) {N}_{2} ( {t}_{4} ) .$$

The apparatus is caused to determine the coefficients α₁¹⁰, β₁¹⁰, γ₁₂¹⁰, α₂¹⁰, β₂¹⁰, and γ₂₁¹⁰ by finding the values those best fit the equations below:

$$\frac{d [ {N}_{1} ( {t}_{1} ) / 2 ]}{dt} = {α}_{1}^{1} \frac{{N}_{1} ( {t}_{1} )}{2} - {{β}_{1}^{1} [ \frac{{N}_{1} ( {t}_{1} )}{2} ]}^{2} - {γ}_{12}^{1} [ {N}_{2} ( {t}_{1} ) - \frac{{N}_{1} ( {t}_{1} )}{2} ] \frac{{N}_{1} ( {t}_{1} )}{2} ;$$

$$\frac{d [ {N}_{1} ( {t}_{2} ) / 2 ]}{dt} = {α}_{1}^{1} \frac{{N}_{1} ( {t}_{2} )}{2} - {{β}_{1}^{1} [ \frac{{N}_{1} ( {t}_{2} )}{2} ]}^{2} - {γ}_{12}^{1} [ {N}_{2} ( {t}_{2} ) - \frac{{N}_{1} ( {t}_{2} )}{2} ] \frac{{N}_{1} ( {t}_{2} )}{2} ;$$

$$\frac{d [ {N}_{1} ( {t}_{3} ) / 2 ]}{dt} = {α}_{1}^{1} \frac{{N}_{1} ( {t}_{3} )}{2} - {{β}_{1}^{1} [ \frac{{N}_{1} ( {t}_{3} )}{2} ]}^{2} - {γ}_{12}^{1} [ {N}_{2} ( {t}_{3} ) - \frac{{N}_{1} ( {t}_{3} )}{2} ] \frac{{N}_{1} ( {t}_{3} )}{2} ;$$

$$\frac{d [ {N}_{1} ( {t}_{4} ) / 2 ]}{dt} = {α}_{1}^{1} \frac{{N}_{1} ( {t}_{4} )}{2} - {{β}_{1}^{1} [ \frac{{N}_{1} ( {t}_{4} )}{2} ]}^{2} - {γ}_{12}^{1} [ {N}_{2} ( {t}_{4} ) - \frac{{N}_{1} ( {t}_{4} )}{2} ] \frac{{N}_{1} ( {t}_{4} )}{2} ;$$

$$\frac{d [ {N}_{2} ( {t}_{1} ) - {N}_{1} ( {t}_{1} ) / 2 ]}{dt} = {α}_{2}^{1} [ {N}_{2} ( {t}_{1} ) - \frac{{N}_{1} ( {t}_{1} )}{2} ] - {{β}_{2}^{1} [ {N}_{2} ( {t}_{1} ) - \frac{{N}_{1} ( {t}_{1} )}{2} ]}^{2} - {γ}_{21}^{1} \frac{{N}_{1} ( {t}_{1} )}{2} [ {N}_{2} ( {t}_{1} ) - \frac{{N}_{1} ( {t}_{1} )}{2} ] ;$$

$$\frac{d [ {N}_{2} ( {t}_{2} ) - {N}_{1} ( {t}_{2} ) / 2 ]}{dt} = {α}_{2}^{1} [ {N}_{2} ( {t}_{2} ) - \frac{{N}_{1} ( {t}_{2} )}{2} ] - {{β}_{2}^{1} [ {N}_{2} ( {t}_{2} ) - \frac{{N}_{1} ( {t}_{2} )}{2} ]}^{2} - {γ}_{21}^{1} \frac{{N}_{1} ( {t}_{2} )}{2} [ {N}_{2} ( {t}_{2} ) - \frac{{N}_{1} ( {t}_{2} )}{2} ] ;$$

$$\frac{d [ {N}_{2} ( {t}_{3} ) - {N}_{1} ( {t}_{3} ) / 2 ]}{dt} = {α}_{2}^{1} [ {N}_{2} ( {t}_{3} ) - \frac{{N}_{1} ( {t}_{3} )}{2} ] - {{β}_{2}^{1} [ {N}_{2} ( {t}_{3} ) - \frac{{N}_{1} ( {t}_{3} )}{2} ]}^{2} - {γ}_{21}^{1} \frac{{N}_{1} ( {t}_{3} )}{2} [ {N}_{2} ( {t}_{3} ) - \frac{{N}_{1} ( {t}_{3} )}{2} ] ;$$

$$\frac{d [ {N}_{2} ( {t}_{4} ) - {N}_{1} ( {t}_{4} ) / 2 ]}{dt} = {α}_{2}^{1} [ {N}_{2} ( {t}_{4} ) - \frac{{N}_{1} ( {t}_{4} )}{2} ] - {{β}_{2}^{1} [ {N}_{2} ( {t}_{4} ) - \frac{{N}_{1} ( {t}_{4} )}{2} ]}^{2} - {γ}_{21}^{1} \frac{{N}_{1} ( {t}_{4} )}{2} [ {N}_{2} ( {t}_{4} ) - \frac{{N}_{1} ( {t}_{4} )}{2} ] .$$

For the second set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₂₁¹ . . . γ₂₁¹², they are determined with a subset of the samples of the examination results by an apparatus having at least one non-transitory computer-readable medium and at least one processor. For example, the apparatus determines the second set coefficients may be determined with N₁(t₁), N₁(t₂), N₁(t₃), N₂(t₁), N₂(t₂), N₂(t₃), with N₁(t₁), N₁(t₃), N₁(t₄), N₂(t₁), N₂(t₃), N₂(t₄), with N₁(t₂), N₁(t₃), N₁(t₄), N₂(t₂), N₂(t₃), N₂(t₄), or with other subset of the samples of the examination results. Similar to the first set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₂₁¹ . . . γ₂₁¹², the apparatus determines the second set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₂₁¹ . . . γ₂₁¹² according to Equation (a).

One variance can be determined from α₁¹ in the first and second sets of coefficients. One variance can be determined from β₁¹ in the first and second sets of coefficients. One variance can be determined from γ₁₂¹ in the first and second sets of coefficients. One variance can be determined from 4 in the first and second sets of coefficients. One variance can be determined from β₂¹ in the first and second sets of coefficients. One variance can be determined from γ₂₁¹ in the first and second sets of coefficients. Thus, for the first possible case, there are six variances for the coefficients (i.e., Var(α₁¹), Var(β₁¹), Var(γ₁₂¹), Var(α₂¹), Var(β₂¹), and Var(γ₂₁¹)), and the variance sum for the first cases is the summation of these six variances (i.e., Var(α₁¹)+Var(β₁¹)+Var(γ₁₂¹)+Var(α₂¹)+Var(β₂¹)+Var(γ₂₁¹)). Similarly, for each one of the other eleven possible cases, there are six variances for the coefficients, and the variance sum for each case is the summation of these six variances (i.e., Var(α₁²)+Var(β₁²)+Var(γ₁₂²)+Var(α₂²)+Var(β₂²)+Var(γ₂₁²), . . . . , Var(α₁¹²)+Var(β₁¹²)+Var(γ₁₂¹²)+Var(α₂¹²)+Var(β₂¹²)+Var(γ₂₁¹²)).

Through comparisons between the variance sums for the 12 cases by the apparatus having at least one non-transitory computer-readable medium and at least one processor, the smallest variance sum can be determined by the apparatus. The apparatus determines the case with the smallest variance sum as the best guess. Thus, the apparatus determines the type of PR between SGA₁ and SGA₂ according to the cases with the smallest variance sum. The apparatus determines whether CN is involved SGAᵢ, SGA₂, or both according to the cases with the smallest variance sum.

In some embodiments, an apparatus having at least one non-transitory computer-readable medium and at least one processor determines the third set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₂₁¹ . . . γ₂₁¹² with another subset of the samples of the examination results. For example, if the apparatus determines the second set of coefficients with N₁(t₁), N₁(t₂), N₁(t₃), N₂(t₁), N₂(t₂), N₂(t₃), the apparatus determines the third set of coefficients with N₁(t₁), N₁(t₃), N₁(t₄), N₂(t₁), N₂(t₃), N₂(t₄), or with N₁(t₂), N₁(t₃), N₁(t₄) N₂(t₂), N₂(t₃), N₂(t₄).

In some embodiments, an apparatus having at least one non-transitory computer-readable medium and at least one processor determines the fourth the fourth set of coefficients α₁¹ . . . α₁¹², β₁¹ . . . β₁¹², γ₁₂¹ . . . γ₁₂¹², α₂¹ . . . α₂¹², β₂¹ . . . β₂¹², and γ₂₁¹ . . . γ₂₁¹², with another subset of the samples of the examination results. For example, if the apparatus determines the second set of coefficients with N₁(t₁), N₁(t₂), N₁(t₃), N₂(t₁), N₂(t₂), N₂(t₃), and if the apparatus determines the third set of coefficients with N₁(t₁), N₁(t₃), N₁(t₄), N₂(t₁), N₂(t₃), N₂(t₄), the apparatus determines the fourth set of coefficients with N₁(t₂), N₁(t₃), N₁(t₄), N₂(t₂), N₂(t₃), N₂(t₄).

In some embodiments that the third and fourth sets coefficients are determined by an apparatus having at least one non-transitory computer-readable medium and at least one processor, one variance can be determined from α₁¹ in the first to fourth sets of coefficients; one variance can be determined from β₁¹ in the first to fourth sets of coefficients; one variance can be determined from γ₁₂¹ in the first to fourth sets of coefficients; one variance can be determined from α₂¹ in the first to fourth sets of coefficients; one variance can be determined from β₂¹ in the first to fourth sets of coefficients; and one variance can be determined from γ₂₁¹ in the first to fourth sets of coefficients. Thus, for the first possible case, there are six variances for the coefficients (i.e., Var(α₂¹), Var(β₁¹), Var(γ₁₂¹), Var(α₂¹), Var(β₂¹), and Var(γ₂₁¹)), and the variance sum for the first case is the summation of these six variances (i.e., Var(α₁¹)+Var(β₁¹)+Var(γ₁₂¹)+Var(α₂¹)+Var(β₂¹)+Var(γ₂₁¹)). Similarly, for each one of the other eleven possible cases, there are six variances for the coefficients, and the variance sum for each case is the summation of these six variances. Similarly, the fifth set of coefficients and forth can be determined, and the variance sum for each one of the 12 cases is determined accordingly.

Through comparisons between the variance sums for 12 cases by the apparatus having at least one non-transitory computer-readable medium and at least one processor, the smallest variance sum can be determined. The apparatus determines the case with the smallest variance sum as the best guess. Thus, according to the best guess, the apparatus determines the type of PR between SGA₁ and SGA₂ and whether CN is involved SGA₁, SGA₂, or both.

For SGA₁ and SGA₃, the type of PR and whether CN is involved can be determined through the method or algorithm similar to that for SGA₁ and SGA₂. For SGA₂ and SGA₃, the type of PR and whether CN is involved can be determined through the method or algorithm similar to that for SGA₁ and SGA₂.

When observing three SGAs, through the above method or algorithm, the type of PR and whether CN is involved can be determined by reviewing 36 (i.e., 3*12) cases. When observing four SGAs, through the above method or algorithm, the type of PR and whether CN is involved can be determined by reviewing 72 (i.e., 6*12) cases. In general, when observing n SGAs, through the above method or algorithm, the type of PR and whether CN is involved can be determined by reviewing C₂ⁿ×12 cases. Through the above method or algorithm, when observing n SGAs, the complexity is O(n!). The complexity of the above method of algorithm (i.e., O(n!)) is much lower than that of the previous method or algorithm(i.e., O(cⁿ), where c is a constant).

Back to the example of observing three SGAs (i.e., SGA₁ to SGA₃) by four examinations, for each possible pair of the three SGAs, after 12 possible cases are reviewed and verified by the “sub-sample verification,” the type of PR and whether CN is involved are determined. For an example that SGA₁ and SGA₂ involves CN, SGA₁ to SGA₂ are parent-child type PR, SGA₁ to SGA₃ are parent-child type PR, and SGA₂ to SGA₃ are sibling type PR. At time t₁, the accurate numbers of cancer cells harboring SGA₁, SGA₂, and SGA₃ (i.e., Ń₁(t₁), Ń₂(t₁), and Ń₃(t₁)) are

$${1}_{} ( {t}_{1} ) = \frac{{N}_{1} ( {t}_{1} )}{2} - \frac{{N}_{2} ( {t}_{1} )}{2} - {N}_{3} ( {t}_{1} ) , {2}_{} ( {t}_{1} ) = \frac{{N}_{2} ( {t}_{1} )}{2} ,$$

and Ń₃(t₁)=N₃(t₁) Accordingly, the accurate numbers of cancer cells harboring SGA₁, SGA₂, and SGA₃ at times t₁ to t₄ (i.e., Ń₁(t₁) to Ń₁(t₄), Ń₂(t₁) to Ń₂(t₄), and Ń₃(t₁) to Ń₃(t₄)) can be determined.

For another example that SGA₁ and SGA₂ involves CN, SGA₁ to SGA₂ are parent-child type PR, SGA₁ to SGA₃ are parent-child type PR, and SGA₂ to SGA₃ are parent-child type PR. At time t₁, the accurate number of cancer cells harboring SGA₁, SGA₂, and SGA₃ (i.e., Ń₁(t₁), Ń₂(t₁), and Ń₃(t₁)) are

$${1}_{} ( {t}_{1} ) = \frac{{N}_{1} ( {t}_{1} )}{2} - \frac{{N}_{2} ( {t}_{1} )}{2} , {2}_{} ( {t}_{1} ) = \frac{{N}_{2} ( {t}_{1} )}{2} - {N}_{3} ( {t}_{1} ) ,$$

and Ń₃(t₁)=N₃(t₁). The reasons why N₃(t₁) is not subtracted from Ń₁(t₁) is that

$$\frac{{N}_{2} ( {t}_{1} )}{2}$$

already includes Ń₃(t₁) (i.e., N₃(t₁)). Accordingly, the accurate numbers of cancer cells harboring SGA₁, SGA₂, and SGA₃ at times t₁ to t₄ (i.e., Ń₁(t₁) to Ń₁(t₄), Ń₂(t₁) to Ń₂(t₄), and Ń₃(t₁) to Ń₃(t₄)) can be determined.

The apparatus having at least one non-transitory computer-readable medium and at least one processor is caused to determine time derivatives of the number of cancer cells harboring SGA₁ to SGA₃ at times t₁, t₂, and t₃ based on the accurate numbers of cancer cells harboring SGA₁, SGA₂, and SGA₃ (i.e., Ń₁(t₁) to Ń₁(t₄), Ń₂(t₁) to Ń₂(t₄), and Ń₃(t₁) to Ń₃(t₄)). For example,

$$\frac{{1}_{} ( {t}_{1} )}{dt}$$

can be regarded as the velocity of growth of cancer cells those harbor SGA₁ at time t₁ or the instantaneous velocity of growth of cancer cells those harbor SGA₁ at time t₁. According to Equation (a), the apparatus is caused to determine the coefficients α₁, α₂, α₃, β₁, β₂, β₃, γ₁₂, γ₁₃, γ₂₁, γ₂₃, γ₃₁, and γ₃₂ (the set of prediction coefficients) (e.g., using the minimization of sum of squared errors or minimization of absolute percentage errors). For example, the apparatus is caused to determine the prediction coefficients α₁, α₂, α₃, β₁, β₂, β₃, γ₁₂, γ₁₃, γ₁₃, γ₂₁, γ₂₃, γ₃₁ and γ₃₂ by finding the values those best fit the equations below:

$${\frac{d ( {t}_{1} )}{dt} = {α}_{1} {1}_{} ( {t}_{1} ) - {{β}_{1} [ {1}_{} ( {t}_{1} ) ]}^{2} - {γ}_{12} {2}_{} ( {t}_{1} ) {1}_{} ( {t}_{1} ) - {γ}_{13} {3}_{} ( {t}_{1} ) {1}_{} ( {t}_{1} ) ; \\ \frac{d ( {t}_{2} )}{dt} = {α}_{1} {1}_{} ( {t}_{2} ) - {{β}_{1} [ {1}_{} ( {t}_{2} ) ]}^{2} - {γ}_{12} {2}_{} ( {t}_{3} ) {1}_{} ( {t}_{2} ) - {γ}_{13} {3}_{} ( {t}_{2} ) {1}_{} ( {t}_{2} ) ; \\ \frac{d ( {t}_{3} )}{dt} = {α}_{1} {1}_{} ( {t}_{3} ) - {β}_{1} {1}_{} ( {t}_{3} ) ]}^{2} - {γ}_{12} {2}_{} ( {t}_{3} ) {1}_{} ( {t}_{3} ) - {γ}_{13} {3}_{} ( {t}_{3} ) {1}_{} ( {t}_{3} ) ;$$

$$\frac{d ( {t}_{4} )}{dt} = {α}_{1} {1}_{} ( {t}_{4} ) - {{β}_{1} [ {1}_{} ( {t}_{4} ) ]}^{2} - {γ}_{12} {2}_{} ( {t}_{4} ) {1}_{} ( {t}_{4} ) - {γ}_{13} {3}_{} ( {t}_{4} ) {1}_{} ( {t}_{4} ) ;$$

$$\frac{d ( {t}_{1} )}{dt} = {α}_{2} {2}_{} ( {t}_{1} ) - {{β}_{2} [ {2}_{} ( {t}_{1} ) ]}^{2} - {γ}_{12} {1}_{} ( {t}_{1} ) {2}_{} ( {t}_{1} ) - {γ}_{23} {3}_{} ( {t}_{1} ) {2}_{} ( {t}_{1} ) ;$$

$$\frac{d ( {t}_{2} )}{dt} = {α}_{2} {2}_{} ( {t}_{2} ) - {{β}_{2} [ {2}_{} ( {t}_{2} ) ]}^{2} - {γ}_{21} {1}_{} ( {t}_{2} ) {2}_{} ( {t}_{2} ) - {γ}_{23} {3}_{} ( {t}_{2} ) {2}_{} ( {t}_{2} ) ;$$

$$\frac{d ( {t}_{3} )}{dt} = {α}_{2} {2}_{} ( {t}_{3} ) - {{β}_{2} [ {2}_{} ( {t}_{3} ) ]}^{2} - {γ}_{21} {1}_{} ( {t}_{3} ) {2}_{} ( {t}_{3} ) - {γ}_{23} {3}_{} ( {t}_{3} ) {2}_{} ( {t}_{3} ) ;$$

$$\frac{d {2}_{} ( {t}_{4} )}{dt} = {α}_{2} {2}_{} ( {t}_{4} ) - {{β}_{2} [ {2}_{} ( {t}_{4} ) ]}^{2} - {γ}_{21} {1}_{} ( {t}_{4} ) {2}_{} ( {t}_{4} ) - {γ}_{23} {3}_{} ( {t}_{4} ) {2}_{} ( {t}_{4} ) ;$$

$$\frac{d {3}_{} ( {t}_{1} )}{dt} = {α}_{3} {3}_{} ( {t}_{1} ) - {{β}_{3} [ {3}_{} ( {t}_{1} ) ]}^{2} - {γ}_{31} {1}_{} ( {t}_{1} ) {3}_{} ( {t}_{1} ) - {γ}_{32} {2}_{} ( {t}_{1} ) {3}_{} ( {t}_{1} ) ;$$

$$\frac{d {3}_{} ( {t}_{2} )}{dt} = {α}_{3} {3}_{} ( {t}_{2} ) - {{β}_{3} [ {3}_{} ( {t}_{2} ) ]}^{2} - {γ}_{31} {1}_{} ( {t}_{2} ) {3}_{} ( {t}_{2} ) - {γ}_{32} {2}_{} ( {t}_{2} ) {3}_{} ( {t}_{2} ) ;$$

$$\frac{d {3}_{} ( {t}_{3} )}{dt} = {α}_{3} {3}_{} ( {t}_{3} ) - {{β}_{2} [ {3}_{} ( {t}_{3} ) ]}^{2} - {γ}_{31} {1}_{} ( {t}_{3} ) {3}_{} ( {t}_{3} ) - {γ}_{32} {2}_{} ( {t}_{3} ) {3}_{} ( {t}_{3} ) ;$$

$$\frac{d {3}_{} ( {t}_{4} )}{dt} = {α}_{3} {3}_{} ( {t}_{4} ) - {{β}_{3} [ {3}_{} ( {t}_{4} ) ]}^{2} - {γ}_{31} {1}_{} ( {t}_{4} ) {3}_{} ( {t}_{4} ) - {γ}_{32} {2}_{} ( {t}_{4} ) {3}_{} ( {t}_{4} ) .$$

After the coefficients α₁, α₂, α₃, β₁, β₂, β₃, γ₁₂, γ₁₃, γ₂₁, γ₂₃, γ₃₁, and γ₃₂ are determined, the differentiation equations that describe the velocity of growth of cancer cell harboring SGA₁, SGA₂ and SGA₃ are given:

$$\frac{d {1}_{} ( t )}{dt} = {α}_{1} {1}_{} ( t ) - {{β}_{1} [ {1}_{} ( t ) ]}^{2} - {γ}_{12} {2}_{} ( t ) {1}_{} ( t ) - {γ}_{13} {3}_{} ( t ) {1}_{} ( t ) ;$$

$$\frac{d {2}_{} ( t )}{dt} = {α}_{2} {2}_{} ( t ) - {{β}_{2} [ {2}_{} ( t ) ]}^{2} - {γ}_{21} {1}_{} ( t ) {2}_{} ( t ) - {γ}_{23} {3}_{} ( t ) {2}_{} ( t ) ;$$

$$and$$

$$\frac{d {3}_{} ( t )}{dt} = {α}_{3} {3}_{} ( t ) - {{β}_{2} [ {3}_{} ( t ) ]}^{2} - {γ}_{31} {1}_{} ( t ) {3}_{} ( t ) - {γ}_{32} {2}_{} ( t ) {3}_{} ( t ) .$$

### Method to Forecast the Dynamics of Cancer Cell Number Under the Current Treatment

The dynamics of the cell number and the competition interactions of cancer cells those harbor the SGAs are described by the equation (b). The accurate CN and PR of SGAs are determined by above methods. The values of the parameters α, β, and γ are estimated with the observed dynamics of the number of cancer cell those harbor the SGAs. Based on the equation (b) and the values of the parameters α, β, and γ, a forward-time simulation can be used to generate the forecast of the future dynamics of cancer cell number under the current treatment.

In one embodiment, the present disclosure provides a method to forecast the dynamics of the cancer cell number under the current treatment, comprising the steps of:

(a) measuring multiple signals of cancer cells harboring SGAs corresponding multiple time points, wherein each signal represents the cancer cell number harboring a specific SGA at a time point; and
(b) obtaining the value of the per capital growth rate (α), the value of the parameter for sensitivity to the intraspecific competition (β), and the value of the parameter for sensitivity to the interspecific competition (γ) of cancer cells those harbor the SGAs by applying statistical parameter estimation methods to estimate the values of the parameters of the equation (b); and
(c) Based on the following equation (equation (b)) and the values of the parameters α, β, γ, a forward-time simulation can be used to generate the forecast of the future dynamics of the cancer cell number under the current treatment.
wherein Nᵢ(t) is the number of cancer cells those harbor SGAᵢ at timepoint t; αᵢ is the per capita growth rate of cancer cells those harbor SGAᵢ; βᵢ is the parameter for sensitivity to the intraspecific competition of cancer cells those harbor SGAᵢ; γᵢⱼ is the parameter for sensitivity to the interspecific competition of cancer cells those harbor SGAᵢ when competing with cancer cells those harbor SGAⱼ; the time derivative of Nᵢ(t) means the velocity of growth of cancer cells those harbor SGAᵢ.

In one embodiment, the present disclosure provides an apparatus to forecast the dynamics of the cancer cell number under the current treatment, comprising:

a receiving module configured to receive multiple signals of cancer cells harboring SGAs corresponding multiple time points, wherein each signal represents the cancer cell number harboring a specific SGA at a time point;

a communication module configured to transmit the multiple values to a database;

a processor coupled with the communication module and configured to obtain the value of the per capital growth rate (α), the value of the parameter for sensitivity to the intraspecific competition (β) and the value of the parameter for sensitivity to the interspecific competition (γ) of cancer cells those harbor the SGAs by applying statistical parameter estimation methods to estimate the values of the parameters of the equation (b). And generate the forecast of the future dynamics of cancer cell number under the current treatment by a forward-time simulation based on the values of the parameters α, β, γ, and the following differentiation equation (equation (b)).

wherein Nᵢ(t) is the number of cancer cells those harbor SGAᵢ at timepoint t; αᵢ is the per capita growth rate of cancer cells those harbor SGAᵢ; βᵢ is the parameter for sensitivity to the intraspecific competition of cancer cells those harbor SGAᵢ; γᵢⱼ is the parameter for sensitivity to the interspecific competition of cancer cells those harbor SGA when competing with cancer cells those harbor SGAⱼ; the time derivative of Nᵢ(t) means the velocity of growth of cancer cells harbor SGAᵢ; and a display module coupled with the processor and configured to display the forecast of the dynamics of the cancer cell number under the current treatment of a specific patient.

### Method to Predict Response to a Subsequent SGA-Targeted Treatment.

In one embodiment, the present disclosure provides a method for prediction of the response to a subsequent SGA-targeted treatment, comprising the steps of:

(a) measuring multiple signals of cancer cells harboring SGAs corresponding multiple time points, wherein each signal represents the cancer cell number harboring a specific SGA at a time point; and
(b) obtaining the value of the per capital growth rate (α), the value of parameter for sensitivity to the intraspecific competition (β) and the value of parameter for sensitivity to the interspecific competition (γ) of cancer cells those harbor the SGAs by applying statistical parameter estimation methods to estimate the values of the parameters of the equation (b); and
(c) the value of the modifying parameter m is obtained by in vitro drug sensitivity studies or in vivo cancer dynamics studies; and
(d) Based on the following equation (equation (c)) and the values of the parameters α, β, γ, and m, a forward-time simulation can be used to generate the forecast of the future dynamics of the cancer cell number under a subsequent treatment.

$$\frac{{dN}_{i} ( t )}{dt} = {m}_{i} {α}_{i} {N}_{i} ( t ) - {{β}_{i} [ {N}_{i} ( t ) ]}^{2} - {\sum }_{j = 1}^{K} {ϒ}_{ij} {N}_{j} ( t ) {N}_{i} ( t ) ,$$

wherein mᵢ represents the modifying parameter of the per capita growth rate of cancer cell that harbor SGAᵢ when an SGA-targeted treatment is given to the patient, and a is determined by the cancer-cell-intrinsic effect of an SGA on the cellular growth and survival.

In one embodiment, the present disclosure provides an apparatus for the prediction of the response to a subsequent SGA-targeted treatment, comprising:

a receiving module configured to receive multiple signals of cancer cells harboring SGAs corresponding multiple time points, wherein each signal represents the cancer cell number harboring a specific SGA at a time point;

a communication module configured to transmit the multiple values to a database;

a processor coupled with the communication module and configured to obtain the value of the per capital growth rate (α), the value of the parameter for sensitivity to the intraspecific competition (β) and the value of the parameter for sensitivity to the interspecific competition (γ) of cancer cells those harbor the SGAs by applying statistical parameter estimation methods to estimate the values of the parameters of the equation (b). And generate the forecast of the future dynamics of the cancer cell number under a subsequent treatment by the forward-time simulation based on the values of the parameters α, β, γ, m and the differentiation equation (equation (c)).

$$\frac{{dN}_{i} ( t )}{dt} = {m}_{i} {α}_{i} {N}_{i} ( t ) - {{β}_{i} [ {N}_{i} ( t ) ]}^{2} - {\sum }_{j = 1}^{K} {ϒ}_{ij} {N}_{j} ( t ) {N}_{i} ( t ) ,$$

wherein mᵢ represents the modifying parameter of the per capita growth rate of cancer cell that harbor SGAᵢ when an SGA-targeted treatment is given to the patient, and a is determined by the cancer-cell-intrinsic effect of an SGA on the cellular growth and survival; and

a display module coupled with the processor and configured to display the predicted response to a specific subsequent treatment of a specific patient.

The prediction of the response to a subsequent SGA-targeted treatment is carried-out with the following method. The dynamics of the cancer cells number under a subsequent treatment is described by the equation(c):

$$\frac{{dN}_{i} ( t )}{dt} = {m}_{i} {α}_{i} {N}_{i} ( t ) - {{β}_{i} [ {N}_{i} ( t ) ]}^{2} - {\sum }_{j = 1}^{K} {ϒ}_{ij} {N}_{j} ( t ) {N}_{i} ( t ) \qquad equation ( c )$$

whereas mᵢ represents the modifying parameter of the per capita growth rate of cancer cell that harbor SGAᵢ under a subsequent SGA-targeted treatment. Given that a is determined by the cancer-cell-intrinsic effect of an SGA on the cellular growth and survival, the anti-cancer treatment and the anti-cancer immunity, a subsequent SGA-targeted treatment results in a new per capita growth rate mᵢαᵢ.

The value of the modifying parameter, mᵢ, of a particular subsequent SGA-targeted treatment can be derived from in vitro drug sensitivity studies, such as MTT assays or in vivo cancer dynamics studies. Due to the SGA-specific inhibitory effect of an SGA-targeted treatment, an SGA-targeted treatment may specifically inhibit cancer cells those harbor a particular SGA (or SGAs) and leaves cancer cells harboring other SGAs relative uninhibited. So, for an SGA-targeted treatment and a “on-target” SGA, the value of m_{on-target} is relatively small to represent the large inhibitory effect on the growth of cells harboring the on-target SGA, on the other hand, for a “off-target” SGA, a relative large value of m_{off-target} represents the growth of cells is relative uninhibited by the treatment. By applying the corresponding modifying factor m to model effects of a subsequent SGA-targeted treatment on the dynamics of the cancer cell number and the values of the parameters α, β, and γ, the forecast of the dynamics of the cancer cell number under a subsequent treatment can be generated by the equation (c) with a forward-time simulation. The response of the tumor to the treatment can be predicted by comparing the simulation-obtained cancer cell number with the number of cancer cell at baseline. Thus, the optimal treatment strategy that results in an adequate control of the cancer cell number can be discovered.

For an example of observing three SGAs, through the above method or algorithm, the differentiation equations for a patient that describe the velocity of growth of cancer cell harboring SGA₁, SGA₂ and SGA₃ are given:

$$\frac{{1}_{} ( t )}{dt} = {α}_{1} {1}_{} ( t ) - {{β}_{1} [ {1}_{} ( t ) ]}^{2} - {γ}_{12} {2}_{} ( t ) {1}_{} ( t ) - {γ}_{13} {3}_{} ( t ) {1}_{} ( t ) ;$$

$$\frac{{2}_{} ( t )}{dt} = {α}_{2} {2}_{} ( t ) - {{β}_{2} [ {2}_{} ( t ) ]}^{2} - {γ}_{21} {1}_{} ( t ) {2}_{} ( t ) - {γ}_{23} {3}_{} ( t ) {2}_{} ( t ) ;$$

$$and$$

$$\frac{{3}_{} ( t )}{dt} = {α}_{3} {3}_{} ( t ) - {{β}_{2} [ {3}_{} ( t ) ]}^{2} - {γ}_{31} {1}_{} ( t ) {3}_{} ( t ) - {γ}_{32} {2}_{} ( t ) {3}_{} ( t ) .$$

If a particular subsequent SGA-targeted treatment is applied to the patient, then the differentiation equations for the patient that describe the velocity of growth of cancer cell harboring SGA₁, SGA₂ and SGA₃ are written as:

$$\frac{{1}_{} ( t )}{dt} = {m}_{1} {α}_{1} {1}_{} ( t ) - {{β}_{1} [ {1}_{} ( t ) ]}^{2} - {γ}_{12} {2}_{} ( t ) {1}_{} ( t ) - {γ}_{13} {3}_{} ( t ) {1}_{} ( t ) ;$$

$$\frac{{2}_{} ( t )}{dt} = {m}_{1} {α}_{2} {2}_{} ( t ) - {{β}_{2} [ {2}_{} ( t ) ]}^{2} - {γ}_{21} {1}_{} ( t ) {2}_{} ( t ) - {γ}_{23} {3}_{} ( t ) {2}_{} ( t ) ;$$

$$and$$

$$\frac{{3}_{} ( t )}{dt} = {m}_{1} {α}_{3} {3}_{} ( t ) - {{β}_{2} [ {3}_{} ( t ) ]}^{2} - {γ}_{31} {1}_{} ( t ) {3}_{} ( t ) - {γ}_{32} {2}_{} ( t ) {3}_{} ( t ) .$$

The coefficients m₁, m₂, and m₃ can be determined from in vitro drug sensitivity studies, such as MTT assays or in vivo cancer dynamics studies. The coefficient m₁ indicates a relative value of the velocity of growth of cancer cell harboring SGA₁ when the particular subsequent SGA-targeted treatment is applied. The coefficient m₂ indicates a relative value of the velocity of growth of cancer cell harboring SGA₂ when the particular subsequent SGA-targeted treatment is applied. The coefficient m₃ indicates a relative value of the velocity of growth of cancer cell harboring SGA₃ when the particular subsequent SGA-targeted treatment is applied. If the particular subsequent SGA-targeted treatment targets SGA₁, m₁ may be smaller than m₂ and m₃. The coefficient mᵢ is a ratio. The coefficient mᵢ is determined from experimental data after scale normalization.

### Method to Improve the Accuracy of Prediction of Treatment Response and Establish the Database of Cancer Dynamics

An accurate prediction of the response to a subsequent treatment depends explicitly on applying the accurate modifying parameter, m, in the equation (c). It is reasonable to assume that the modifying parameter m is determined by the patient-specific and the treatment-specific factors. The data of the dynamics of the relative abundance of SGAs, the dynamics of the cancer cell number, the anti-cancer treatment given to the patient, the biochemical and biophysical index of patients, the demographics of patients obtained in clinical studies will be used to establish a database of the cancer dynamics. Based on the database of the cancer dynamics, the determinants of the modifying parameter m will be defined and the predicted value of m for a specific patient under a specific treatment could be derived by predictive models.

The apparatus and methods of the present disclosure can improve the accuracy of the prediction of the treatment response and establish the database of the cancer dynamics.

The examples are offered for illustrative purposes only and are not intended to limit the scope of the present invention in any way.

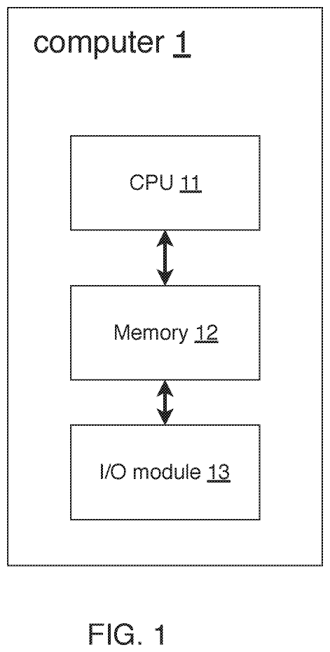

The present disclosure provides a system for processing cancer cells prediction. Referring to FIG. 1, the system essentially comprises a computer 1. The computer 1 may be a server. In some embodiments, the computer 1 comprises a central processing unit (CPU) 11 (or a processor), a memory 12 (or a non-transitory computer-readable medium), and an input/output module 13. The CPU 11, memory 12, and the input/output module 13 are in communication. In some embodiments, the memory 12 has computer executable instructions stored therein. The memory 12 and the computer executable instructions are configured to, with the CPU 11, cause the computer 1 to perform various operations, methods, or algorithms as disclosed in the present disclosure.

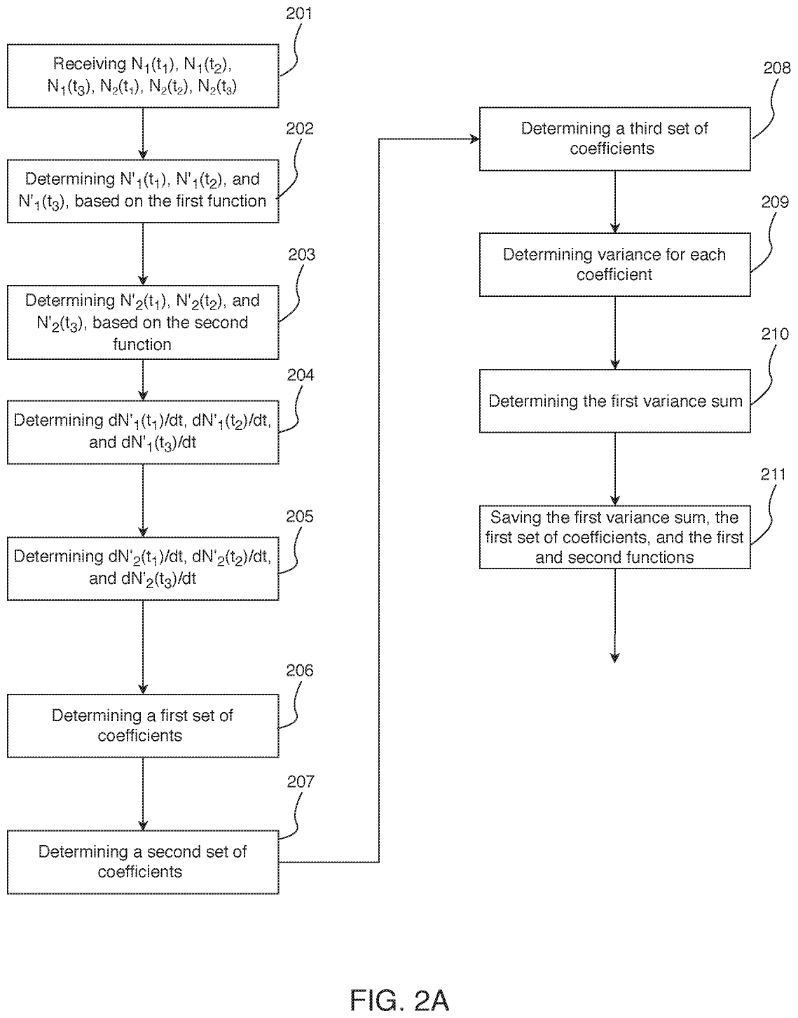

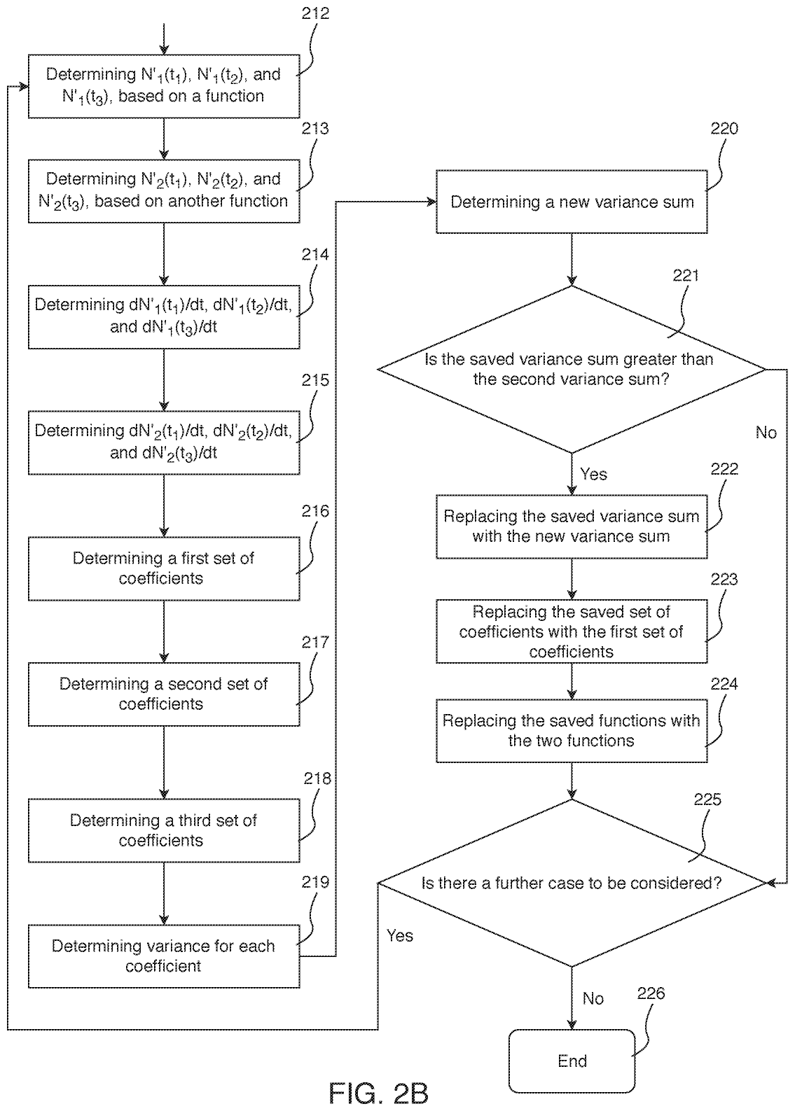

FIGS. 2A and 2B are flow charts according to some embodiments of the present disclosure. At operation 201, the computer 1 receives the count (i.e., number) of cancer cells harboring SGA₁ at time t₁, the count of cancer cells harboring SGA₁ at time t₂, the count of cancer cells harboring SGA₁ at time t₃, the count of cancer cells harboring SGA₂ at time t₁, the count of cancer cells harboring SGA₂ at time t₂, and the count of cancer cells harboring SGA₂ at time t₃. In some embodiments, the computer 1 may receive the counts of cancer cells at further times (e.g., times t₄, t₅, t₆, and forth) for processing. For simplicity, the counts at times t₁, t₂, t₃, are discussed here.

When considering at least one confounding factors between two SGAs, the corrected count of the cancer cells harboring SGA₁ may be represented as a function of the count of the cancer cells harboring SGA₁ and the count of the cancer cells harboring SGA₂. The corrected count of the cancer cells harboring SGA₂ may be represented as another function of the count of the cancer cells harboring SGA₁ and the count of the cancer cells harboring SGA₂. For example, when considering PR and CN between two SGAs, in one possible case, the corrected count of the cancer cells harboring SGA₁ (i.e., Ń₁(t)) may be represented as

$${{N}^{'}}_{1} ( t ) = {N}_{1} ( t ) - \frac{{N}_{2} ( t )}{2} ,$$

and the corrected count of the cancer cells harboring SGA₂ (i.e., Ń₂(t)) may be represented as

$${{N}^{'}}_{2} ( t ) = \frac{{N}_{2} ( t )}{2} .$$

The functions for corrected count of cancer cells harboring a SGA can be determined by the cases in which the at least one confounding factor is considered.

At operation 202, Ń₁(t₁), Ńᵢ(t₂), N₁(t₃) is determined based the first function and N₁(t₁), N₁(t₂), N₁(t₃), N₂(t₁), N₂(t₂), and N₂(t₃). The first function is determined by and associated with the first case that the at least one confounding factor is considered. At operation 203, Ń₂(t₁), Ń₂(t₂), Ń₂(t₃) is determined based the second function and N₁(t₁), N₁(t₂), N₁(t₃), N₂(t₁), N₂(t₂), and N₂(t₃). The second function is determined by and associated with the first case that the at least one confounding factor is considered.

At operation 204, the time derivatives of the corrected counts of cancer cells harboring SGA₁ at t₁ to t₃

$$( i . e . , \frac{d {1}_{} ( {t}_{1} )}{dt} , \frac{d {1}_{} ( {t}_{2} )}{dt} , \frac{d {1}_{} ( {t}_{3} )}{dt}$$

are determined.

At operation 205, the time derivatives of the corrected counts of cancer cells harboring SGA₂ at t₁ to t₃

$$( i . e . , \frac{d {2}_{} ( {t}_{1} )}{dt} , \frac{d {2}_{} ( {t}_{2} )}{dt} , \frac{d {2}_{} ( {t}_{3} )}{dt}$$

are determined.

At operation 206, a first set of coefficients α₁, β₁, γ₁₂, α₂, β₂, and γ₂₁ are determined by finding the values (e.g., using the minimization of sum of squared errors or minimization of absolute percentage errors) those best fit the equations below:

$$\frac{d {1}_{} ( {t}_{1} )}{dt} = {α}_{1} {1}_{} ( {t}_{1} ) - {{β}_{1} [ {1}_{} ( {t}_{1} ) ]}^{2} - {γ}_{12} {2}_{} ( {t}_{1} ) {1}_{} ( {t}_{1} ) ;$$

$$\frac{d {1}_{} ( {t}_{2} )}{dt} = {α}_{1} {1}_{} ( {t}_{2} ) - {{β}_{1} [ {1}_{} ( {t}_{2} ) ]}^{2} - {γ}_{12} {2}_{} ( {t}_{2} ) {1}_{} ( {t}_{2} ) ;$$

$$\frac{d {1}_{} ( {t}_{3} )}{dt} = {α}_{1} {1}_{} ( {t}_{3} ) - {{β}_{1} [ {1}_{} ( {t}_{3} ) ]}^{2} - {γ}_{12} {2}_{} ( {t}_{3} ) {1}_{} ( {t}_{3} ) ;$$

$$\frac{d {2}_{} ( {t}_{1} )}{dt} = {α}_{2} {2}_{} ( {t}_{1} ) - {{β}_{2} [ {2}_{} ( {t}_{1} ) ]}^{2} - {γ}_{21} {1}_{} ( {t}_{1} ) {2}_{} ( {t}_{1} ) ;$$

$$\frac{d {2}_{} ( {t}_{2} )}{dt} = {α}_{2} {2}_{} ( {t}_{2} ) - {{β}_{2} [ {2}_{} ( {t}_{2} ) ]}^{2} - {γ}_{21} {1}_{} ( {t}_{2} ) {2}_{} ( {t}_{2} ) ; and$$

$$\frac{d {2}_{} ( {t}_{3} )}{dt} = {α}_{2} {2}_{} ( {t}_{3} ) - {{β}_{2} [ {2}_{} ( {t}_{3} ) ]}^{2} - {γ}_{21} {N}_{1} ( {t}_{3} ) {2}_{} ( {t}_{3} ) .$$

At operation 207, a second set of coefficients α₁, β₁, γ₁₂, α₂, β₂, and γ₂₁ are determined with a subset of the samples. In some embodiments, at operation 207, a second coefficients α₁, β₁, γ₁₂, α₂, β₂, and γ₂₁ are determined by finding the values (e.g., using the minimization of sum of squared errors or minimization of absolute percentage errors) those best fit the equations below:

$$\frac{d {{N}^{'}}_{1} ( {t}_{1} )}{dt} = {α}_{1} {{N}^{'}}_{1} ( {t}_{1} ) - {{β}_{1} [ {{N}^{'}}_{1} ( {t}_{1} ) ]}^{2} - {γ}_{12} {{N}^{'}}_{2} ( {t}_{1} ) {{N}^{'}}_{1} ( {t}_{1} ) ;$$

$$\frac{d {{N}^{'}}_{1} ( {t}_{2} )}{dt} = {α}_{1} {{N}^{'}}_{1} ( {t}_{2} ) - {{β}_{1} [ {{N}^{'}}_{1} ( {t}_{2} ) ]}^{2} - {γ}_{12} {{N}^{'}}_{2} ( {t}_{2} ) {{N}^{'}}_{1} ( {t}_{2} ) ;$$

$$\frac{d {{N}^{'}}_{2} ( {t}_{1} )}{dt} = {α}_{2} {{N}^{'}}_{2} ( {t}_{1} ) - {{β}_{2} [ {{N}^{'}}_{2} ( {t}_{1} ) ]}^{2} - {γ}_{21} {{N}^{'}}_{1} ( {t}_{1} ) {{N}^{'}}_{2} ( {t}_{1} ) ; and$$

$$\frac{d {{N}^{'}}_{2} ( {t}_{2} )}{dt} = {α}_{2} {{N}^{'}}_{2} ( {t}_{2} ) - {{β}_{2} [ {{N}^{'}}_{2} ( {t}_{2} ) ]}^{2} - {γ}_{21} {{N}^{'}}_{1} ( {t}_{2} ) {{N}^{'}}_{2} ( {t}_{2} ) .$$

At operation 208, a third set of coefficients α₁, β₁, γ₁₂, α₂, β₂, and γ₂₁ are determined with another subset of the samples. In some embodiments, at operation 208, a second coefficients α₁, β₁, γ₁₂, α₂, β₂, and γ₂₁ are determined by finding the values (e.g., using the minimization of sum of squared errors or minimization of absolute percentage errors) those best fit the equations below:

$$\frac{d {{N}^{'}}_{1} ( {t}_{1} )}{dt} = {α}_{1} {{N}^{'}}_{1} ( {t}_{1} ) - {{β}_{1} [ {{N}^{'}}_{1} ( {t}_{1} ) ]}^{2} - {γ}_{12} {{N}^{'}}_{2} ( {t}_{1} ) {{N}^{'}}_{1} ( {t}_{1} ) ;$$

$$\frac{d {{N}^{'}}_{1} ( {t}_{3} )}{dt} = {α}_{1} {{N}^{'}}_{1} ( {t}_{2} ) - {{β}_{1} [ {{N}^{'}}_{1} ( {t}_{3} ) ]}^{2} - {γ}_{12} {{N}^{'}}_{2} ( {t}_{3} ) {{N}^{'}}_{1} ( {t}_{3} ) ;$$

$$\frac{d {{N}^{'}}_{2} ( {t}_{1} )}{dt} = {α}_{2} {{N}^{'}}_{2} ( {t}_{1} ) - {{β}_{2} [ {{N}^{'}}_{2} ( {t}_{1} ) ]}^{2} - {γ}_{21} {{N}^{'}}_{1} ( {t}_{1} ) {{N}^{'}}_{2} ( {t}_{1} ) ; and$$

$$\frac{d {{N}^{'}}_{2} ( {t}_{3} )}{dt} = {α}_{2} {{N}^{'}}_{2} ( {t}_{3} ) - {{β}_{2} [ {{N}^{'}}_{2} ( {t}_{3} ) ]}^{2} - {γ}_{21} {{N}^{'}}_{1} ( {t}_{3} ) {{N}^{'}}_{2} ( {t}_{3} ) .$$

At operation 209, a variance of α₁ is determined based on the α₁ in the first, second, and third set of coefficients. A variance of β₁ is determined based on the β₁ in the first, second, and third set of coefficients. A variance of γ₁₂ is determined based on the γ₁₂ in the first, second, and third set of coefficients. A variance of α₂ is determined based on the α₂ in the first, second, and third set of coefficients. A variance of β₂ is determined based on the β₂ in the first, second, and third set of coefficients. A variance of γ₂₁ is determined based on the γ₂₁ in the first, second, and third set of coefficients.

At operation 210, the first variance sum is determined by summing the variances of α₁, β₁, γ₁₂, α₂, β₂, and γ₂₁.

At operation 211, the first variance sum, the first set of coefficients, the first function, and the second function are saved in the memory 12.

At operation 212, Ń₁(t₁), Ń₁(t₂), Ń₁(t₃) is determined based a function and N₁(t₁), N₁(t₂), N₁(t₃), N₂(t₁), N₂(t₂), and N₂(t₃). The function is determined by and associated with the second case that the at least one confounding factor is considered.

At operation 213, Ń₂(t₁), Ń₂(t₂), Ń₂(t₃) is determined based another function and N₁(t₁), N₁(t₂), N₁(t₃), N₂(t₁), N₂(t₂), and N₂(t₃). The function is determined by and associated with the second case that the at least one confounding factor is considered.

At operation 214, the time derivatives of the corrected counts of cancer cells harboring SGA₁ at t₁ to t₃

$$( i . e . , \frac{d {{N}^{'}}_{1} ( {t}_{1} )}{dt} , \frac{d {{N}^{'}}_{1} ( {t}_{2} )}{dt} , \frac{d {{N}^{'}}_{1} ( {t}_{3} )}{dt}$$

are determined.

At operation 215, the time derivatives of the corrected counts of cancer cells harboring SGA₂ at t₁ to t₃

$$( i . e . , \frac{d {{N}^{'}}_{2} ( {t}_{1} )}{dt} , \frac{d {{N}^{'}}_{2} ( {t}_{2} )}{dt} , \frac{d {{N}^{'}}_{2} ( {t}_{3} )}{dt}$$

are determined.

At operation 216, a first set of coefficients α₁, β₁, γ₁₂, α₂, β₂, and γ₂₁ are determined by finding the values (e.g., using the minimization of sum of squared errors or minimization of absolute percentage errors) those best fit the equations below:

$$\frac{d {{N}^{'}}_{1} ( {t}_{1} )}{dt} = {α}_{1} {{N}^{'}}_{1} ( {t}_{1} ) - {{β}_{1} [ {{N}^{'}}_{1} ( {t}_{1} ) ]}^{2} - {γ}_{12} {{N}^{'}}_{2} ( {t}_{1} ) {{N}^{'}}_{1} ( {t}_{1} ) ;$$

$$\frac{d {{N}^{'}}_{1} ( {t}_{2} )}{dt} = {α}_{1} {{N}^{'}}_{1} ( {t}_{2} ) - {{β}_{1} [ {{N}^{'}}_{1} ( {t}_{2} ) ]}^{2} - {γ}_{12} {{N}^{'}}_{2} ( {t}_{2} ) {{N}^{'}}_{1} ( {t}_{2} ) ;$$

$$\frac{d {{N}^{'}}_{1} ( {t}_{3} )}{dt} = {α}_{1} {{N}^{'}}_{1} ( {t}_{3} ) - {{β}_{1} [ {{N}^{'}}_{1} ( {t}_{3} ) ]}^{2} - {γ}_{12} {{N}^{'}}_{2} ( {t}_{3} ) {{N}^{'}}_{1} ( {t}_{3} ) ;$$

$$\frac{d {{N}^{'}}_{2} ( {t}_{1} )}{dt} = {α}_{2} {{N}^{'}}_{2} ( {t}_{1} ) - {{β}_{2} [ {{N}^{'}}_{2} ( {t}_{1} ) ]}^{2} - {γ}_{21} {{N}^{'}}_{1} ( {t}_{1} ) {{N}^{'}}_{2} ( {t}_{1} ) ;$$

$$\frac{d {{N}^{'}}_{2} ( {t}_{2} )}{dt} = {α}_{2} {{N}^{'}}_{2} ( {t}_{2} ) - {{β}_{2} [ {{N}^{'}}_{2} ( {t}_{2} ) ]}^{2} - {γ}_{21} {{N}^{'}}_{1} ( {t}_{2} ) {{N}^{'}}_{2} ( {t}_{2} ) ; and$$

$$\frac{d {{N}^{'}}_{2} ( {t}_{3} )}{dt} = {α}_{2} {{N}^{'}}_{2} ( {t}_{3} ) - {{β}_{2} [ {{N}^{'}}_{2} ( {t}_{3} ) ]}^{2} - {γ}_{21} {{N}^{'}}_{1} ( {t}_{3} ) {{N}^{'}}_{2} ( {t}_{3} ) .$$

At operation 217, a second set of coefficients α₁, β₁, γ₁₂, α₂, β₂, and γ₂₁ are determined with a subset of the samples. In some embodiments, at operation 207, a second coefficients α₁, β₁, γ₁₂, α₂, β₂, and γ₂₁ are determined by finding the values (e.g., using the minimization of sum of squared errors or minimization of absolute percentage errors) those best fit the equations below:

$$\frac{d {{N}^{'}}_{1} ( {t}_{1} )}{dt} = {α}_{1} {{N}^{'}}_{1} ( {t}_{1} ) - {{β}_{1} [ {{N}^{'}}_{1} ( {t}_{1} ) ]}^{2} - {γ}_{12} {{N}^{'}}_{2} ( {t}_{1} ) {{N}^{'}}_{1} ( {t}_{1} ) ;$$

$$\frac{d {{N}^{'}}_{1} ( {t}_{2} )}{dt} = {α}_{1} {{N}^{'}}_{1} ( {t}_{2} ) - {{β}_{1} [ {{N}^{'}}_{1} ( {t}_{2} ) ]}^{2} - {γ}_{12} {{N}^{'}}_{2} ( {t}_{1} ) {{N}^{'}}_{1} ( {t}_{1} ) ;$$

$$\frac{d {{N}^{'}}_{2} ( {t}_{1} )}{dt} = {α}_{2} {{N}^{'}}_{2} ( {t}_{1} ) - {{β}_{2} [ {{N}^{'}}_{2} ( {t}_{1} ) ]}^{2} - {γ}_{21} {{N}^{'}}_{1} ( {t}_{1} ) {{N}^{'}}_{2} ( {t}_{1} ) ; and$$

$$\frac{d {{N}^{'}}_{2} ( {t}_{2} )}{dt} = {α}_{2} {{N}^{'}}_{2} ( {t}_{2} ) - {{β}_{2} [ {{N}^{'}}_{2} ( {t}_{2} ) ]}^{2} - {γ}_{21} {{N}^{'}}_{1} ( {t}_{2} ) {{N}^{'}}_{2} ( {t}_{2} ) .$$

At operation 218, a third set of coefficients α1, β1, γ12, α2, β2, and γ21 are determined with another subset of the samples. In some embodiments, at operation 208, a second coefficients α₁, β₁, γ₁₂, α₂, β₂, and γ₂₁ are determined by finding the values (e.g., using the minimization of sum of squared errors or minimization of absolute percentage errors) those best fit the equations below:

$$\frac{d {{N}^{'}}_{1} ( {t}_{1} )}{dt} = {α}_{1} {{N}^{'}}_{1} ( {t}_{1} ) - {{β}_{1} [ {{N}^{'}}_{1} ( {t}_{1} ) ]}^{2} - {γ}_{12} {{N}^{'}}_{2} ( {t}_{1} ) {{N}^{'}}_{1} ( {t}_{1} ) ;$$

$$\frac{d {{N}^{'}}_{1} ( {t}_{3} )}{dt} = {α}_{1} {{N}^{'}}_{1} ( {t}_{3} ) - {{β}_{1} [ {{N}^{'}}_{1} ( {t}_{3} ) ]}^{2} - {γ}_{12} {{N}^{'}}_{2} ( {t}_{3} ) {{N}^{'}}_{1} ( {t}_{3} ) ;$$

$$\frac{d {{N}^{'}}_{2} ( {t}_{1} )}{dt} = {α}_{2} {{N}^{'}}_{2} ( {t}_{1} ) - {{β}_{2} [ {{N}^{'}}_{2} ( {t}_{1} ) ]}^{2} - {γ}_{21} {{N}^{'}}_{1} ( {t}_{1} ) {{N}^{'}}_{2} ( {t}_{1} ) ; and$$

$$\frac{d {{N}^{'}}_{2} ( {t}_{3} )}{dt} = {α}_{2} {{N}^{'}}_{2} ( {t}_{3} ) - {{β}_{2} [ {{N}^{'}}_{2} ( {t}_{3} ) ]}^{2} - {γ}_{21} {{N}^{'}}_{1} ( {t}_{3} ) {{N}^{'}}_{2} ( {t}_{3} ) .$$

At operation 219, a variance of α₁ is determined based on the α₁ in the first, second, and third set of coefficients. A variance of β₁ is determined based on the β₁ in the first, second, and third set of coefficients. A variance of γ₁₂ is determined based on the γ₁₂ in the first, second, and third set of coefficients. A variance of α₂ is determined based on the α₂ in the first, second, and third set of coefficients. A variance of β₂ is determined based on the β₂ in the first, second, and third set of coefficients. A variance of γ₂₁ is determined based on the γ₂₁ in the first, second, and third set of coefficients.

At operation 220, a new variance sum is determined by summing the variances of α₁, β₁, γ₁₂, α₂, β₂, and γ₂₁.

If the variance sum is small, the selected case is close to the best guess. At operation 221, if the saved variance sum is greater than the second variance sum, operations 222, 223, and 224 are performed. if the saved variance sum is not greater than the second variance sum, operations 225 are performed.

At operation 222, the saved variance sum is replaced with the new variance sum.

At operation 223, the saved set of coefficients is replaced with the first set of coefficients determined at operation 216.

At operation 224, the saved functions are replaced with the two functions used at operations 212 and 213.

At operation 225, if there is a further case to be considered, operation 212 is performed. The functions used in operations 212 and 213 are determined by and associated with the further case (e.g., third case or forth) that the at least one confounding factor is considered.

Through operations disclosed in FIGS. 2A and 2B, the matched (or best guess) type of the at least one confounding factor between two SGAs can be determined by the saved two functions because the saved two functions are associated with one of the cases that the at least one confounding factor is considered

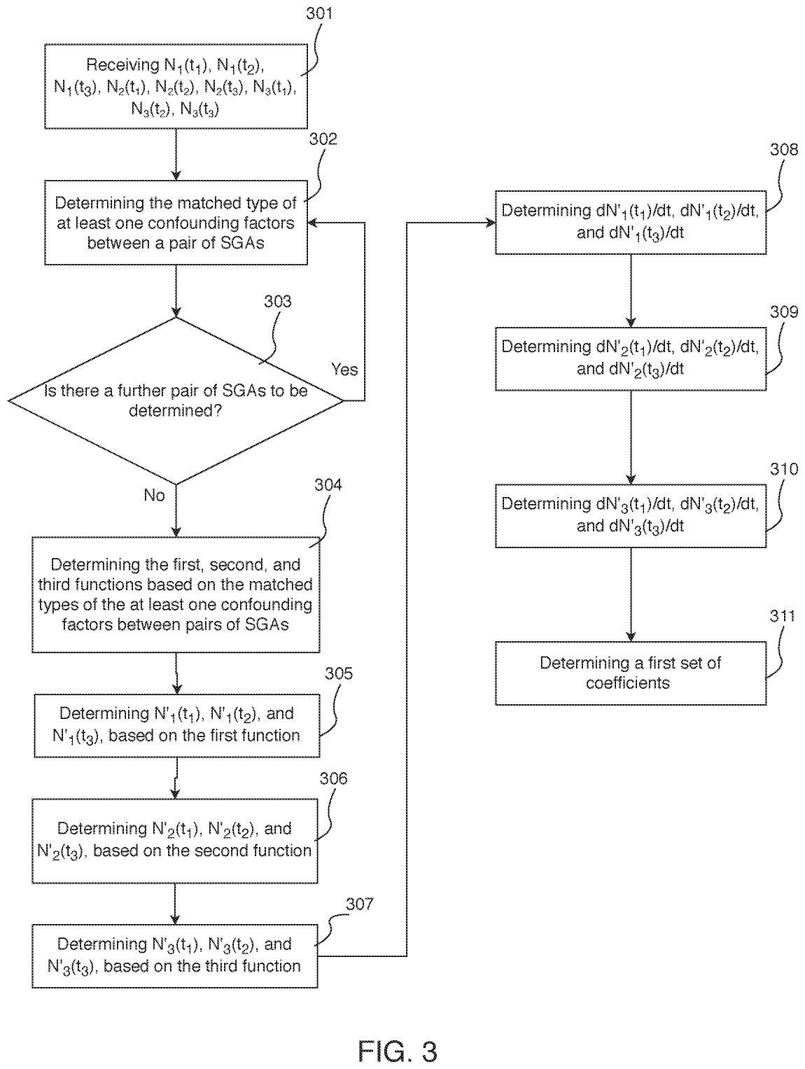

At operation 301, the computer 1 receives the count (i.e., number) of cancer cells harboring SGA₁ at time t₁, the count of cancer cells harboring SGA₁ at time t₂, the count of cancer cells harboring SGA₁ at time t₃, the count of cancer cells harboring SGA₂ at time t₁, the count of cancer cells harboring SGA₂ at time t₂, the count of cancer cells harboring SGA₂ at time t₃, the count of cancer cells harboring SGA₃ at time t₁, the count of cancer cells harboring SGA₃ at time t₂, the count of cancer cells harboring SGA₃ at time t₃. In some embodiments, the computer 1 may receive the counts of cancer cells at further times (e.g., times t₄, t₅, t₆, and forth) for processing. For simplicity, the counts at times t₁, t₂, t₃, are discussed here. In some embodiments, the operations disclosed in FIG. 3 can be used for four SGAs or more.

At operation 302, for a pair of SGAs (e.g., SGA₁ and SGA₂), the matched type of at least one confounding factors is determined through the operations disclosed in FIGS. 2A and 2B.

At operation 303, if there is a further pair of SGAs whose matched type of at least one has to be determined (e.g., SGA₁ and SGA₃ or SGA₁ and SGA₃), operation 302 is performed for the further pair of SGAs. If there is no further pair of SGAs whose matched type of at least one has to be determined, operation 304 is performed.

At operation 304, the first, second, and third functions for the accurate (or corrected) counts of cancer cells harboring SGA₁, SGA₂, and SGA₃ are determined based on the matched type of the at least one confounding factors between pairs of SGAs.

For an example that SGA₁ and SGA₂ involves CN, SGA₁ to SGA₂ are parent-child type PR, SGA₁ to SGA₃ are parent-child type PR, and SGA₂ to SGA₃ are sibling type PR. At time t₁, the first, second, and third functions for the accurate counts of cancer cells harboring SGA₁, SGA₂, and SGA₃ (i.e., Ń₁(t₁), Ń₂(t₁), and Ń₃(t₁)) are

$${{N}^{'}}_{1} ( {t}_{1} ) = \frac{{N}_{1} ( {t}_{1} )}{2} - \frac{{N}_{2} ( {t}_{1} )}{2} - {N}_{3} ( {t}_{1} ) , {{N}^{'}}_{2} ( {t}_{1} ) = \frac{{N}_{2} ( {t}_{1} )}{2} ,$$

and Ń₃ (t₁)=Ń₃(t₁).

For another example that SGA₁ and SGA₂ involves CN, SGA₁ to SGA₂ are parent-child type PR, SGA₁ to SGA₃ are parent-child type PR, and SGA₂ to SGA₃ are parent-child type PR. At time t₁, the first, second, and third functions for the accurate counts of cancer cells harboring SGA₁, SGA₂, and SGA₃ (i.e., Ń₁(t₁), Ń₂(t₁), and Ń₃(t₁)) are

$${{N}^{'}}_{1} ( {t}_{1} ) = \frac{{N}_{1} ( {t}_{1} )}{2} - \frac{{N}_{2} ( {t}_{1} )}{2} , {{N}^{'}}_{2} ( {t}_{1} ) = \frac{{N}_{2} ( {t}_{1} )}{2} - {N}_{3} ( {t}_{1} ) ,$$

and Ń₃(t₁)=N₃(t₁). The reasons why N₃(t₁) is not subtracted from Ń₁(t₁) is that

$$\frac{{N}_{2} ( {t}_{1} )}{2}$$

already includes Ń₃(t₁) i.e., N₃(t₁)).

At operation 305, Ń₁(t₁), Ń₁(t₂), Ń₁(t₃) is determined based the first function and N₁(t₁)d, N₁(t₂), N₁(t₃), N₂(t₁), N₂(t₂), N₂(t₃), N₃(t₁), N₃(t₂), and N₃(t₃).

At operation 306, Ń₂(t₁), Ń₂(t₂), Ń₂(t₃) is determined based the second function and N₁(t₁), N₁(t₂), N₁(t₃), N₂(t₁), N₂(t₂), N₂(t₃), N₃(t₁) N₃(t₂), and N₃ (t₃).

At operation 307, Ń₃(t₁), Ń₃(t₂), Ń₃(t₃) is determined based the second function and N₁(t₁), N₁(t₂), N₁(t₃), N₂(t₁) N₂(t₂), N₂(t₃), N₃(t₁), N₃(t₂), and N₃ (t₃).

At operation 308, the time derivatives of the corrected counts of cancer cells harboring SGA₁ at t₁ to t₃

$$( i . e . , \frac{d {{N}^{'}}_{1} ( {t}_{1} )}{dt} , \frac{d {{N}^{'}}_{1} ( {t}_{2} )}{dt} , \frac{d {{N}^{'}}_{1} ( {t}_{3} )}{dt}$$

are determined.

At operation 309, the time derivatives of the corrected counts of cancer cells harboring SGA₂ at t₁ to t₃

$$( i . e . , \frac{d {{N}^{'}}_{2} ( {t}_{1} )}{dt} , \frac{d {{N}^{'}}_{2} ( {t}_{2} )}{dt} , \frac{d {{N}^{'}}_{2} ( {t}_{3} )}{dt}$$

are determined.

At operation 310, the time derivatives of the corrected counts of cancer cells harboring SGA₃ at t₁ to t₃

$$( i . e . , \frac{d {{N}^{'}}_{3} ( {t}_{1} )}{dt} , \frac{d {{N}^{'}}_{3} ( {t}_{2} )}{dt} , \frac{d {{N}^{'}}_{3} ( {t}_{3} )}{dt}$$

are determined.

At operation 311, a first set of coefficients α₁, α₂, α₃, β₁, β₂, β₃, γ₁₂, γ₁₃, γ₂₁, γ₂₃, γ₃₁, and γ₃₂ are determined by finding the values (e.g., using the minimization of sum of squared errors or minimization of absolute percentage errors) those best fit the equations below:

$${\frac{d {{N}^{′}}_{1} ( {t}_{1} )}{dt} = {α}_{1} {{N}^{′}}_{1} ( {t}_{1} ) - {{β}_{1} [ {{N}^{′}}_{1} ( {t}_{1} ) ]}^{2} - {γ}_{12} {{N}^{′}}_{2} ( {t}_{1} ) {{N}^{′}}_{1} ( {t}_{1} ) - {γ}_{13} {{N}^{′}}_{3} ( {t}_{1} ) {{N}^{′}}_{1} ( {t}_{1} ) ; \\ \frac{d {{N}^{′}}_{1} ( {t}_{2} )}{dt} = {α}_{1} {{N}^{′}}_{1} ( {t}_{2} ) - {{β}_{1} [ {N}_{1} ( {t}_{2} ) ]}^{2} - {γ}_{12} {{N}^{′}}_{2} ( {t}_{3} ) {{N}^{′}}_{1} ( {t}_{2} ) - {γ}_{13} {{N}^{′}}_{3} ( {t}_{2} ) {{N}^{′}}_{1} ( {t}_{2} ) ; \\ \frac{d {{N}^{′}}_{1} ( {t}_{3} )}{dt} = {α}_{1} {{N}^{′}}_{1} ( {t}_{3} ) - {β}_{1} {N}^{′} ( {t}_{3} ) ]}^{2} - {γ}_{12} {{N}^{′}}_{2} ( {t}_{3} ) {{N}^{′}}_{1} ( {t}_{3} ) - {γ}_{13} {{N}^{′}}_{3} ( {t}_{3} ) {{N}^{′}}_{1} ( {t}_{3} ) ;$$

$$\frac{d {{N}^{′}}_{2} ( {t}_{1} )}{dt} = {α}_{2} {{N}^{′}}_{2} ( {t}_{2} ) - {{β}_{2} [ {{N}^{′}}_{2} ( {t}_{1} ) ]}^{2} - {γ}_{21} {{N}^{′}}_{1} ( {t}_{1} ) {{N}^{′}}_{2} ( {t}_{1} ) - {γ}_{23} {{N}^{′}}_{3} ( {t}_{1} ) {{N}^{′}}_{2} ( {t}_{1} ) ;$$

$$\frac{d {{N}^{′}}_{2} ( {t}_{2} )}{dt} = {α}_{2} {{N}^{′}}_{2} ( {t}_{2} ) - {{β}_{2} [ {{N}^{′}}_{2} ( {t}_{2} ) ]}^{2} - {γ}_{21} {{N}^{′}}_{1} ( {t}_{2} ) {{N}^{′}}_{2} ( {t}_{2} ) - {γ}_{23} {{N}^{′}}_{3} ( {t}_{2} ) {{N}^{′}}_{2} ( {t}_{2} ) ;$$

$$\frac{d {{N}^{′}}_{2} ( {t}_{3} )}{dt} = {α}_{2} {{N}^{′}}_{2} ( {t}_{3} ) - {{β}_{2} [ {{N}^{′}}_{2} ( {t}_{3} ) ]}^{2} - {γ}_{21} {{N}^{′}}_{1} ( {t}_{3} ) {{N}^{′}}_{2} ( {t}_{3} ) - {γ}_{23} {{N}^{′}}_{3} ( {t}_{3} ) {{N}^{′}}_{2} ( {t}_{3} ) ;$$

$$\frac{d {{N}^{′}}_{3} ( {t}_{1} )}{dt} = {α}_{3} {{N}^{′}}_{3} ( {t}_{1} ) - {{β}_{3} [ {{N}^{′}}_{3} ( {t}_{1} ) ]}^{2} - {γ}_{31} {{N}^{′}}_{1} ( {t}_{1} ) {{N}^{′}}_{3} ( {t}_{1} ) - {γ}_{32} {{N}^{′}}_{2} ( {t}_{1} ) {{N}^{′}}_{3} ( {t}_{1} ) ;$$

$$\frac{d {{N}^{′}}_{3} ( {t}_{2} )}{dt} = {α}_{3} {{N}^{′}}_{3} ( {t}_{2} ) - {{β}_{3} [ {{N}^{′}}_{3} ( {t}_{2} ) ]}^{2} - {γ}_{31} {{N}^{′}}_{1} ( {t}_{2} ) {{N}^{′}}_{3} ( {t}_{2} ) - {γ}_{32} {{N}^{′}}_{2} ( {t}_{2} ) {{N}^{′}}_{3} ( {t}_{2} ) ;$$

$$\frac{d {{N}^{′}}_{3} ( {t}_{3} )}{dt} = {α}_{3} {{N}^{′}}_{3} ( {t}_{3} ) - {{β}_{3} [ {{N}^{′}}_{3} ( {t}_{3} ) ]}^{2} - {γ}_{31} {{N}^{′}}_{1} ( {t}_{3} ) {{N}^{′}}_{3} ( {t}_{3} ) - {γ}_{32} {{N}^{′}}_{2} ( {t}_{3} ) {{N}^{′}}_{3} ( {t}_{3} ) ;$$

What is claimed is:

1. An apparatus, comprising:
at least one non-transitory computer-readable medium having computer executable instructions stored therein; and
at least one processor coupled to the at least one non-transitory computer-readable medium,
wherein the at least one non-transitory computer-readable medium and the computer executable instructions are configured to, with the at least one processor, cause the apparatus to perform following operations:
receiving a first value, a second value, and a third value;
wherein the first value indicates a detected count of cells having a first feature at a first time, the second value indicates a detected count of cells having a first feature at a second time, and the third value indicates a detected count of cells having a first feature at a third time;
receiving a fourth value, a fifth value, and a sixth value;
wherein the fourth value indicates a detected count of cells having a second feature at the first time, the fifth value indicates a detected count of cells having the second feature at the second time, and the sixth value indicates a detected count of cells having the second feature at the third time;
determining a seventh value based on a first function, the first value, and the fourth value;
determining an eighth value based on the first function, the second value, and the fifth value;
determining a ninth value based on the first function, the third value, and the sixth value;
determining a tenth value based on a second function, the first value, and the fourth value;
determining an eleventh value based on the second function, the second value, and the fifth value;
determining a twelfth value based on the second function, the third value, and the sixth value;
determining a first velocity, a second velocity, and a third velocity based on the seventh to ninth values;
determining a fourth velocity, a fifth velocity, and a sixth velocity based on the tenth to twelfth values;
determining a first set of coefficients based on the first to sixth velocities and the seventh to twelfth values;
determining a second set of coefficients based on the first, second, fourth and fifth velocities and the seventh, eighth, tenth, and eleventh values;
for each coefficient of the first set of coefficients, determining a variance with the corresponding coefficient in the second set of coefficients;
determining a first variance sum by summing the variances for the coefficients in the first and second sets of coefficients; and
saving the first variance sum, the first set of coefficients, the first function, and the second function.

2. The apparatus of claim 1, wherein the apparatus is caused to further perform following operations:
determining a third set of coefficients based on the first, third, fourth, and sixth velocities and the seventh, ninth, tenth, and twelfth values;
for each coefficient of the first set of coefficients, determining a variance with the corresponding coefficient in the second set of coefficients and the corresponding coefficient in the third set of coefficients;
determining the first variance sum by summing the variances for the coefficients in the first, second, and third sets of coefficients

3. The apparatus of claim 1, wherein the apparatus is caused to further perform following operations:
determining a thirteenth value based on a third function, the first value, and the fourth value;
determining a fourteenth value based on the third function, the second value, and the fifth value;
determining a fifteenth value based on the third function, the third value, and the sixth value;
determining a sixteenth value based on a fourth function, the first value, and the fourth value;
determining a seventeenth value based on the fourth function, the second value, and the fifth value;
determining an eighteenth value based on the fourth function, the third value, and the sixth value;
determining an seventh velocity, an eighth velocity, and a ninth velocity based on the thirteenth to fifteenth values;
determining a tenth velocity, a eleventh velocity, and a twelfth velocity based on the sixteenth to eighteenth values;
determining a third set of coefficients based on the seventh to twelfth velocities and the thirteenth to eighteenth values;
determining a fourth set of coefficients based on the seventh, eighth, tenth, and eleventh velocities and the thirteenth, fourteenth, sixteenth, and seventeenth values;
for each coefficient of the third set of coefficients, determining a variance with the corresponding coefficient in the fourth set of coefficients;
determining a second variance sum by summing the variances for the coefficients in the third and fourth sets of coefficients;
comparing the saved variance sum and the second variance sum; and
if the saved variance sum is greater than the second variance sum, replacing the saved variance sum with the second variance sum, replacing the saved set of coefficients with the third set of coefficients, and replacing the saved functions with the third function and the fourth function.

4. The apparatus of claim 3, wherein the apparatus is caused to further perform following operations:
determining a nineteenth value based on a fifth function, the first value, and the fourth value;
determining a twentieth value based on the fifth function, the second value, and the fifth value;
determining a twenty-first value based on the fifth function, the third value, and the sixth value;
determining a twenty-second value based on a sixth function, the first value, and the fourth value;
determining a twenty-third value based on the sixth function, the second value, and the fifth value;
determining a twenty-fourth value based on the sixth function, the third value, and the sixth value;
determining a thirteenth velocity, a fourteenth velocity, and a fifteenth velocity based on the nineteenth to twenty-first values;
determining a sixteenth velocity, a seventeenth velocity, and an eighteenth velocity based on the twenty-second to twenty-fourth values;
determining a fifth set of coefficients based on the thirteenth to eighteenth velocities and the nineteenth to twenty-fourth values;
determining a sixth set of coefficients based on the fourteenth, fifteenth, seventeenth, and eighteenth velocities and the twentieth, twenty-first, twenty-third, and twenty-fourth values;
for each coefficient of the fifth set of coefficients, determining a variance with the corresponding coefficient in the sixth set of coefficients;
determining a third variance sum by summing the variances for the coefficients in the fifth and sixth sets of coefficients;
comparing the saved variance sum and the third variance sum; and
if the saved variance sum is greater than the third variance sum, replacing the saved variance sum with the third variance sum, replacing the saved set of coefficients with the fifth set of coefficients, and replacing the saved functions with the fifth function and the sixth function.

5. The apparatus of claim 3, wherein the saved coefficients includes α₁, β₁, γ₁₂, α₂, β₂, and γ₂₁, and the saved set of coefficients matches the equations below:
$$\frac{{dN}_{1} ( t )}{dt} = {α}_{1} {N}_{1} ( t ) - {{β}_{1} [ {N}_{1} ( t ) ]}^{2} - {γ}_{12} {N}_{2} ( t ) {N}_{1} ( t ) and$$
$$\frac{{dN}_{2} ( t )}{dt} = {α}_{2} {N}_{2} ( t ) - {{β}_{2} [ {N}_{2} ( t ) ]}^{2} - {γ}_{21} {N}_{1} ( t ) {N}_{2} ( t ) ,$$
where N₁(t) refers to a count of cells having the first feature at time t, N₂(t) refers to a count of cells having the second feature at time t,
$$\frac{{dN}_{1} ( t )}{dt}$$
refers to the time derivative of N₁(t), and
$$\frac{{dN}_{2} ( t )}{dt}$$
refers to the time derivative of N₂ (t).

6. The apparatus of claim 1, wherein the first and second functions are associated with at least one confounding factor.

7. The apparatus of claim 6, wherein the at least one confounding factor includes the phylogenetic relationship (PR) and the copy number (CN).

8. The apparatus of claim 3, wherein the apparatus is caused to further perform following operation:
determining a matched type of a confounding factor between the first and second features based on the saved functions.

9. An apparatus, comprising:
at least one non-transitory computer-readable medium having computer executable instructions stored therein; and
at least one processor coupled to the at least one non-transitory computer-readable medium,
wherein the at least one non-transitory computer-readable medium and the computer executable instructions are configured to, with the at least one processor, cause the apparatus to perform following operations:
receiving a first value, a second value, and a third value;
wherein the first value indicates a detected count of cells having a first feature at a first time, the second value indicates a detected count of cells having a first feature at a second time, and the third value indicates a detected count of cells having a first feature at a third time;
receiving a fourth value, a fifth value, and a sixth value;
wherein the fourth value indicates a detected count of cells having a second feature at the first time, the fifth value indicates a detected count of cells having the second feature at the second time, and the sixth value indicates a detected count of cells having the second feature at the third time;
receiving a seventh value, an eighth value, and a ninth value;
wherein the seventh value indicates a detected count of cells having a third feature at the first time, the eighth value indicates a detected count of cells having the third feature at the second time, and the ninth value indicates a detected count of cells having the third feature at the third time;
performing the operations of the apparatus of claim 8 to determine a first matched type of a confounding factor between the first and second features;
performing the operations of the apparatus of claim 8 to determine a second matched type of the confounding factor between the first and third features;
performing the operations of the apparatus of claim 8 to determine a third matched type of the confounding factor between the second and third features;
determining a first function, a second function, and a third function based on the first to third matched type of the confounding factor;
determining a first accurate value based on the first function, the first value, the fourth value, and the seventh value;
determining a second accurate value based on the first function, the second value, the fifth value, and the eighth value;
determining a third accurate value based on the first function, the third value, the sixth value, and the ninth value;
determining a fourth accurate value based on the second function, the first value, the fourth value, and the seventh value;
determining a fifth accurate value based on the second function, the second value, the fifth value, and the eighth value;
determining a sixth accurate value based on the second function, the third value, the sixth value, and the ninth value;
determining a seventh accurate value based on the third function, the first value, the fourth value, and the seventh value;
determining an eighth accurate value based on the third function, the second value, the fifth value, and the eighth value;
determining a ninth accurate value based on the third function, the third value, the sixth value, and the ninth value;
determining a first velocity, a second velocity, and a third velocity based on the first to third accurate values;
determining a fourth velocity, a fifth velocity, and a sixth velocity based on the fourth to sixth accurate values;
determining a seventh velocity, an eighth velocity, and a ninth velocity based on the seventh to ninth accurate values; and
determining a first set of coefficients based on the first to ninth velocities and the first to ninth accurate values.

10. The apparatus of claim 9, wherein the first set of coefficients includes α₁, α₂, α₃, β₁, β₂, β₃, γ₁₂, γ₁₃, γ₂₁, γ₂₃, γ₃₁, and γ₃₂, and the first set of coefficients matches the equations below:
$$\frac{{dN}_{1} ( t )}{dt} = {α}_{1} {N}_{1} ( t ) - {{β}_{1} [ {N}_{1} ( t ) ]}^{2} - {γ}_{12} {N}_{2} ( t ) {N}_{1} ( t ) - {γ}_{13} {N}_{3} ( t ) {N}_{1} ( t ) ;$$
$$\frac{{dN}_{2} ( t )}{dt} = {α}_{2} {N}_{2} ( t ) - {{β}_{2} [ {N}_{2} ( t ) ]}^{2} - {γ}_{21} {N}_{1} ( t ) {N}_{2} ( t ) - {γ}_{23} {N}_{3} ( t ) {N}_{2} ( t ) ; and$$
$$\frac{{dN}_{3} ( t )}{dt} = {α}_{3} {N}_{3} ( t ) - {{β}_{2} [ {N}_{3} ( t ) ]}^{2} - {γ}_{31} {N}_{1} ( t ) {N}_{3} ( t ) - {γ}_{32} {N}_{2} ( t ) {N}_{3} ( t ) ,$$
where N₁(t) refers to a count of cells having the first feature at time t, N₂(t) refers to a count of cells having the second feature at time t, N₃(t) refers to a count of cells having the third feature at time t,
$$\frac{{dN}_{1} ( t )}{dt}$$
refers to the time derivative of N₁(t),
$$\frac{{dN}_{3} ( t )}{dt}$$
refers to the time derivative of N₂(t), and
$$\frac{{dN}_{3} ( t )}{dt}$$
refers to the time derivative of N₃(t).

11. A method, comprising:
receiving a first value, a second value, and a third value;
wherein the first value indicates a detected count of cells having a first feature at a first time, the second value indicates a detected count of cells having a first feature at a second time, and the third value indicates a detected count of cells having a first feature at a third time;
receiving a fourth value, a fifth value, and a sixth value;
wherein the fourth value indicates a detected count of cells having a second feature at the first time, the fifth value indicates a detected count of cells having the second feature at the second time, and the sixth value indicates a detected count of cells having the second feature at the third time;
determining a seventh value based on a first function, the first value, and the fourth value;
determining an eighth value based on the first function, the second value, and the fifth value;
determining a ninth value based on the first function, the third value, and the sixth value;
determining a tenth value based on a second function, the first value, and the fourth value;
determining an eleventh value based on the second function, the second value, and the fifth value;
determining a twelfth value based on the second function, the third value, and the sixth value;
determining a first velocity, a second velocity, and a third velocity based on the seventh to ninth values;
determining a fourth velocity, a fifth velocity, and a sixth velocity based on the tenth to twelfth values;
determining a first set of coefficients based on the first to sixth velocities and the seventh to twelfth values;
determining a second set of coefficients based on the first, second, fourth and fifth velocities and the seventh, eighth, tenth, and eleventh values;
for each coefficient of the first set of coefficients, determining a variance with the corresponding coefficient in the second set of coefficients;
determining a first variance sum by summing the variances for the coefficients in the first and second sets of coefficients; and
saving the first variance sum, the first set of coefficients, the first function, and the second function.

12. The method of claim 11, further comprising:
determining a third set of coefficients based on the first, third, fourth, and sixth velocities and the seventh, ninth, tenth, and twelfth values;
for each coefficient of the first set of coefficients, determining a variance with the corresponding coefficient in the second set of coefficients and the corresponding coefficient in the third set of coefficients;
determining the first variance sum by summing the variances for the coefficients in the first, second, and third sets of coefficients

13. The method of claim 11, further comprising:
determining a thirteenth value based on a third function, the first value, and the fourth value;
determining a fourteenth value based on the third function, the second value, and the fifth value;
determining a fifteenth value based on the third function, the third value, and the sixth value;
determining a sixteenth value based on a fourth function, the first value, and the fourth value;
determining a seventeenth value based on the fourth function, the second value, and the fifth value;
determining an eighteenth value based on the fourth function, the third value, and the sixth value;
determining an seventh velocity, an eighth velocity, and a ninth velocity based on the thirteenth to fifteenth values;
determining a tenth velocity, a eleventh velocity, and a twelfth velocity based on the sixteenth to eighteenth values;
determining a third set of coefficients based on the seventh to twelfth velocities and the thirteenth to eighteenth values;
determining a fourth set of coefficients based on the seventh, eighth, tenth, and eleventh velocities and the thirteenth, fourteenth, sixteenth, and seventeenth values;
for each coefficient of the third set of coefficients, determining a variance with the corresponding coefficient in the fourth set of coefficients;
determining a second variance sum by summing the variances for the coefficients in the third and fourth sets of coefficients;
comparing the saved variance sum and the second variance sum; and
if the saved variance sum is greater than the second variance sum, replacing the saved variance sum with the second variance sum, replacing the saved set of coefficients with the third set of coefficients, and replacing the saved functions with the third function and the fourth function.

14. The method of claim 13, further comprising:
determining a nineteenth value based on a fifth function, the first value, and the fourth value;
determining a twentieth value based on the fifth function, the second value, and the fifth value;
determining a twenty-first value based on the fifth function, the third value, and the sixth value;
determining a twenty-second value based on a sixth function, the first value, and the fourth value;
determining a twenty-third value based on the sixth function, the second value, and the fifth value;
determining a twenty-fourth value based on the sixth function, the third value, and the sixth value;
determining a thirteenth velocity, a fourteenth velocity, and a fifteenth velocity based on the nineteenth to twenty-first values;
determining a sixteenth velocity, a seventeenth velocity, and an eighteenth velocity based on the twenty-second to twenty-fourth values;
determining a fifth set of coefficients based on the thirteenth to eighteenth velocities and the nineteenth to twenty-fourth values;
determining a sixth set of coefficients based on the fourteenth, fifteenth, seventeenth, and eighteenth velocities and the twentieth, twenty-first, twenty-third, and twenty-fourth values;
for each coefficient of the fifth set of coefficients, determining a variance with the corresponding coefficient in the sixth set of coefficients;
determining a third variance sum by summing the variances for the coefficients in the fifth and sixth sets of coefficients;
comparing the saved variance sum and the third variance sum; and
if the saved variance sum is greater than the third variance sum, replacing the saved variance sum with the third variance sum, replacing the saved set of coefficients with the fifth set of coefficients, and replacing the saved functions with the fifth function and the sixth function.

15. The method of claim 13, wherein the saved coefficients includes α₁, β₁, γ₁₂, α₂, β₂, and γ₂₁, and the saved set of coefficients matches the equations below:
$$\frac{{dN}_{1} ( t )}{dt} = {α}_{1} {N}_{1} ( t ) - {{β}_{1} [ {N}_{1} ( t ) ]}^{2} - {γ}_{12} {N}_{2} ( t ) {N}_{1} ( t ) and$$
$$\frac{{dN}_{2} ( t )}{dt} = {α}_{2} {N}_{2} ( t ) - {{β}_{2} [ {N}_{2} ( t ) ]}^{2} - {γ}_{21} {N}_{1} ( t ) {N}_{2} ( t ) ,$$
where N₁(t) refers to a count of cells having the first feature at time t, N₂(t) refers to a count of cells having the second feature at time t,
$$\frac{{dN}_{1} ( t )}{dt}$$
refers to the time derivative of N₁(t), and
$$\frac{{dN}_{2} ( t )}{dt}$$
refers to the time derivative of N₂(t).

16. The method of claim 11, wherein the first and second functions are associated with at least one confounding factor.

17. The method of claim 16, wherein the at least one confounding factor includes the phylogenetic relationship (PR) and the copy number (CN).

18. The method of claim 13, wherein the apparatus is caused to further perform following operation:
determining a matched type of a confounding factor between the first and second features based on the saved functions.

19. A method, comprising:
receiving a first value, a second value, and a third value;
wherein the first value indicates a detected count of cells having a first feature at a first time, the second value indicates a detected count of cells having a first feature at a second time, and the third value indicates a detected count of cells having a first feature at a third time;
receiving a fourth value, a fifth value, and a sixth value;
wherein the fourth value indicates a detected count of cells having a second feature at the first time, the fifth value indicates a detected count of cells having the second feature at the second time, and the sixth value indicates a detected count of cells having the second feature at the third time;
receiving a seventh value, an eighth value, and a ninth value;
wherein the seventh value indicates a detected count of cells having a third feature at the first time, the eighth value indicates a detected count of cells having the third feature at the second time, and the ninth value indicates a detected count of cells having the third feature at the third time;
performing the method of claim 18 to determine a first matched type of a confounding factor between the first and second features;
performing the method of claim 18 to determine a second matched type of the confounding factor between the first and third features;
performing the method of claim 18 to determine a third matched type of the confounding factor between the second and third features;
determining a first function, a second function, and a third function based on the first to third matched type of the confounding factor;
determining a first accurate value based on the first function, the first value, the fourth value, and the seventh value;
determining a second accurate value based on the first function, the second value, the fifth value, and the eighth value;
determining a third accurate value based on the first function, the third value, the sixth value, and the ninth value;
determining a fourth accurate value based on the second function, the first value, the fourth value, and the seventh value;
determining a fifth accurate value based on the second function, the second value, the fifth value, and the eighth value;
determining a sixth accurate value based on the second function, the third value, the sixth value, and the ninth value;
determining a seventh accurate value based on the third function, the first value, the fourth value, and the seventh value;
determining an eighth accurate value based on the third function, the second value, the fifth value, and the eighth value;
determining a ninth accurate value based on the third function, the third value, the sixth value, and the ninth value;
determining a first velocity, a second velocity, and a third velocity based on the first to third accurate values;
determining a fourth velocity, a fifth velocity, and a sixth velocity based on the fourth to sixth accurate values;
determining a seventh velocity, an eighth velocity, and a ninth velocity based on the seventh to ninth accurate values; and
determining a first set of coefficients based on the first to ninth velocities and the first to ninth accurate values.

20. The apparatus of claim 19, wherein the first set of coefficients includes α₁, α₂, α₃, β₁, β₂, β₃, γ₁₂, γ₁₃, γ₂₁, γ₂₃, γ₃₁, and γ₃₂, and the first set of coefficients matches the equations below:
$$\frac{{dN}_{1} ( t )}{dt} = {α}_{1} {N}_{1} ( t ) - {{β}_{1} [ {N}_{1} ( t ) ]}^{2} - {γ}_{12} {N}_{2} ( t ) {N}_{1} ( t ) - {γ}_{13} {N}_{3} ( t ) {N}_{1} ( t ) ;$$
$$\frac{{dN}_{2} ( t )}{dt} = {α}_{2} {N}_{2} ( t ) - {{β}_{2} [ {N}_{2} ( t ) ]}^{2} - {γ}_{21} {N}_{1} ( t ) {N}_{2} ( t ) - {γ}_{23} {N}_{3} ( t ) {N}_{2} ( t ) ; and$$
$$\frac{{dN}_{3} ( t )}{dt} = {α}_{3} {N}_{3} ( t ) - {{β}_{2} [ {N}_{3} ( t ) ]}^{2} - {γ}_{31} {N}_{1} ( t ) {N}_{3} ( t ) - {γ}_{32} {N}_{2} ( t ) {N}_{3} ( t ) ,$$
where N₁(t) refers to a count of cells having the first feature at time t, N₂(t) refers to a count of cells having the second feature at time t, N₃(t) refers to a count of cells having the third feature at time t,
$$\frac{{dN}_{1} ( t )}{dt}$$
refers to the time derivative of N₁(t),
$$\frac{{dN}_{2} ( t )}{dt}$$
refers to the time derivative of N₂(t), and
$$\frac{{dN}_{3} ( t )}{dt}$$
refers to the time derivative of N₃(t).
